(19) United States
(12) Patent Application Publication
(10) Pub. No.: US 2024/0389377 A1
(43) Pub. Date: Nov. 21, 2024

(54) Light-Emitting Device, Light-Emitting Apparatus, Display Apparatus, Electronic Device, Lighting Device

(71) Applicant: Semiconductor Energy Laboratory Co., Ltd., Kanagawa-ken (JP)

(72) Inventors: Naoaki HASHIMOTO, Sagamihara, Kanagawa (JP); Satoshi SEO, Sagamihara, Kanagawa (JP); Tsunenori SUZUKI, Yokohama, Kanagawa (JP); Hiromi SEO, Sagamihara, Kanagawa (JP)

(73) Assignee: Semiconductor Energy Laboratory Co., Ltd., Kanagawa-ken (JP)

(21) Appl. No.: 18/289,091

(22) PCT Filed: Apr. 28, 2022
(86) PCT No.: PCT/IB2022/053936

§ 371 Date: Oct. 31, 2023

(30) Foreign Application Priority Data
May 13, 2021 (JP) 2021-081420

(51) Int. Cl. H10K 50/16; C07D 401/10; H10K 50/15; H10K 85/60; H10K 101/30

(52) U.S. Cl. CPC H10K 50/16; C07D 401/10; H10K 50/15; H10K 85/6572; H10K 2101/30

(57) ABSTRACT

A novel light-emitting device that is highly convenient, useful, or reliable is provided. The light-emitting device includes a first electrode, a second electrode, a first unit, and a first layer, and the first unit is held between the first electrode and the second electrode and includes a second layer, a third layer, and a fourth layer. The second layer is held between the third layer and the fourth layer, and the second layer contains a light-emitting material. The fourth layer is held between the second layer and the second electrode and contains a first organic compound. The first organic compound has a π-electron deficient heteroaromatic ring skeleton and a π-electron rich heteroaromatic ring skeleton and has a HOMO level higher than or equal to −6.0 eV and lower than or equal to −5.6 eV. The first layer is held between the first electrode and the first unit, is in contact with the first electrode, and contains a second organic compound and a third organic compound.

### TECHNICAL FIELD

One embodiment of the present invention relates to a light-emitting device, a light-emitting apparatus, a display apparatus, an electronic device, or a lighting device.

Note that one embodiment of the present invention is not limited to the above technical field. The technical field of one embodiment of the invention disclosed in this specification relates to an object, a method, or a manufacturing method. Alternatively, one embodiment of the present invention relates to a process, a machine, manufacture, or a composition of matter. Thus, more specifically, examples of the technical field of one embodiment of the present invention disclosed in this specification include a semiconductor device, a display apparatus, a light-emitting apparatus, a power storage device, a memory device, a driving method thereof, and a manufacturing method thereof.

### BACKGROUND ART

Light-emitting devices (organic EL devices) including organic compounds and utilizing electroluminescence (EL) have been put to more practical use. In the basic structure of such light-emitting devices, an organic compound layer containing a light-emitting material (an EL layer) is held between a pair of electrodes. Carriers (holes and electrons) are injected by application of voltage to the element, and recombination energy of the carriers is used, whereby light emission can be obtained from the light-emitting material.

Such light-emitting devices are of self-luminous type and thus have advantages over liquid crystal, such as high visibility and no need for backlight when used in pixels of a display, and are suitable as flat panel display elements. Displays including such light-emitting devices are also highly advantageous in that they can be thin and lightweight. Another feature is an extremely fast response speed.

Since light-emitting layers of such light-emitting devices can be successively formed two-dimensionally, planar light emission can be obtained. This feature is difficult to realize with point light sources typified by incandescent lamps and LEDs or linear light sources typified by fluorescent lamps; thus, the light-emitting devices also have great potential as planar light sources, which can be applied to lighting and the like.

Displays or lighting devices including light-emitting devices are suitable for a variety of electronic devices as described above, and research and development of light-emitting devices has progressed for more favorable characteristics.

In a known light-emitting device, for example, an EL layer includes a first layer, a second layer, a third layer, a light-emitting layer, and a fourth layer sequentially from an anode side, the first layer contains a first organic compound and a second organic compound, the fourth layer contains a seventh organic compound, the first organic compound has an electron-accepting property with respect to the second organic compound, the highest occupied molecular orbital (HOMO) level of the second organic compound is higher than or equal to −5.7 eV and lower than or equal to −5.2 eV, and the seventh organic compound has an electron mobility higher than or equal to 1×10⁻⁷ cm²/Vs and lower than or equal to 5×10⁻⁵ cm²/Vs when the square root of the electric-field strength [V/cm] is 600 (Patent Document 1).

### REFERENCE

### Patent Document

[Patent Document 1] Japanese Published Patent Application No. 2020-96171

### SUMMARY OF THE INVENTION

### Problems to be Solved by the Invention

An object of one embodiment of the present invention is to provide a novel light-emitting device that is highly convenient, useful, or reliable. Another object is to provide a novel light-emitting apparatus that is highly convenient, useful, or reliable. Another object is to provide a novel display apparatus that is highly convenient, useful, or reliable. Another object is to provide a novel electronic device that is highly convenient, useful, or reliable. Another object is to provide a novel lighting device that is highly convenient, useful, or reliable. Another object is to provide a novel light-emitting device, a novel light-emitting apparatus, a novel display apparatus, a novel electronic device, or a novel lighting device.

Note that the description of these objects does not preclude the existence of other objects. Note that one embodiment of the present invention does not have to achieve all the objects. Other objects will be apparent from the description of the specification, the drawings, the claims, and the like, and other objects can be derived from the description of the specification, the drawings, the claims, and the like.

### Means for Solving the Problems

(1) One embodiment of the present invention is a light-emitting device that includes a first electrode, a second electrode, a first unit, and a first layer.

The first unit is held between the first electrode and the second electrode, and the first unit includes a second layer, a third layer, and a fourth layer.

The second layer is held between the third layer and the fourth layer, and the second layer contains a light-emitting material.

The fourth layer is held between the second layer and the second electrode, the fourth layer contains a first organic compound, and the first organic compound has a π-electron deficient heteroaromatic ring skeleton and a π-electron rich heteroaromatic ring skeleton.

The first layer is held between the first electrode and the first unit, and the first layer is in contact with the first electrode. The first layer contains a second organic compound and a third organic compound, and the third organic compound has an electron-accepting property with respect to the second organic compound.

The first layer has a resistivity higher than or equal to 1×10⁴ [Ω·cm] and lower than or equal to 1×10⁷ [Ω·cm].

(2) Another embodiment of the present invention is the above-described light-emitting device in which the first organic compound has a first HOMO level and the first HOMO level is higher than or equal to −6.0 eV and lower than or equal to −5.6 eV.

(3) Another embodiment of the present invention is the above-described light-emitting device in which the first organic compound has a diazine skeleton and a π-electron rich heteroaromatic ring skeleton.

Accordingly, electron transfer from the second electrode to the second layer can be facilitated.

(4) Another embodiment of the present invention is the above-described light-emitting device in which the first organic compound has a π-electron deficient heteroaromatic ring skeleton and a carbazole skeleton.

Accordingly, electron transfer from the second layer to the fourth layer can be facilitated.

(5) Another embodiment of the present invention is the above-described light-emitting device in which the first organic compound is represented by General Formula (G1) below.

[Chemical Formula 1]

D-Ar-E (G1)

In General Formula (G1) above, D represents a substituted or unsubstituted quinoxalinyl group, E represents a substituted or unsubstituted carbazolyl group. Furthermore, Ar represents a substituted or unsubstituted arylene group, and the arylene group has 6 to 13 carbon atoms forming a ring.

Accordingly, electron transfer from the second electrode to the second layer can be facilitated. In addition, hole transfer from the second layer to the fourth layer can be facilitated. Accumulation of holes between the second layer and the fourth layer can be reduced. Accumulation of holes at the interface between the second layer and the fourth layer can be reduced. Consequently, a novel light-emitting device that is highly convenient, useful, or reliable can be provided.

(6) Another embodiment of the present invention is the above-described light-emitting device in which the third organic compound has a lowest unoccupied molecular orbital (LUMO) level lower than or equal to −5.0 eV, the second organic compound has a second HOMO level, and the second HOMO level is higher than or equal to −5.7 eV and lower than or equal to −5.3 eV.

(7) Another embodiment of the present invention is the above-described light-emitting device in which when a square root of electric-field strength [V/cm] is 600, the second organic compound has a hole mobility lower than or equal to 1×10⁻³ cm/Vs.

(8) Another embodiment of the present invention is the above-described light-emitting device in which the first layer has a resistivity higher than or equal to 5×10⁴ [Ω·cm] and lower than or equal to 1×10⁷ [Ω·cm].

(9) Another embodiment of the present invention is the above-described light-emitting device in which the first layer has a resistivity higher than or equal to 1×10⁵ [Ω·cm] and lower than or equal to 1×10⁷ [Ω·cm].

In this manner, hole injection from the first electrode to the first unit can be facilitated. In addition, flow of holes in the first layer can be appropriately inhibited. A phenomenon in which holes flow into an adjacent light-emitting device unintentionally can be inhibited. A crosstalk phenomenon in which an adjacent light-emitting device unintentionally operates can be inhibited. As a result, a novel light-emitting device that is highly convenient or reliable can be provided.

(10) Another embodiment of the present invention is the above-described light-emitting device in which the third layer is held between the first layer and the second layer and the third layer is in contact with the first layer.

In the above-described light-emitting device, the third layer contains a fourth organic compound, the fourth organic compound has a third HOMO level, and the third HOMO level differs from the second HOMO level by higher than or equal to −0.2 eV and lower than or equal to 0 eV.

(11) One embodiment of the present invention is a display apparatus that includes a first light-emitting device and a second light-emitting device.

The first light-emitting device has the above-described structure, and the second light-emitting device is adjacent to the first light-emitting device.

The second light-emitting device includes a third electrode and a fifth layer, and a first gap is between the third electrode and the first electrode.

The fifth layer is held between the third electrode and the second electrode, the fifth layer is in contact with the third electrode, and the fifth layer contains the second organic compound. A second gap is between the fifth layer and the first layer, and the second gap overlaps with the first gap.

(12) Another embodiment of the present invention is a light-emitting apparatus that includes the above-described light-emitting device, and a transistor or a substrate.

(13) Another embodiment of the present invention is a display apparatus that includes the above-described light-emitting device, and a transistor or a substrate.

(14) Another embodiment of the present invention is a lighting device that includes the above-described light-emitting apparatus and a housing.

(15) Another embodiment of the present invention is an electronic device that includes the above-described display apparatus, and a sensor, an operation button, a speaker, or a microphone.

Although a block diagram in which components are classified by their functions and shown as independent blocks is shown in the drawing attached to this specification, it is difficult to completely separate actual components according to their functions and one component can relate to a plurality of functions.

Note that the light-emitting apparatus in this specification includes, in its category, an image display device that uses a light-emitting device. The light-emitting apparatus may also include a module in which a light-emitting device is provided with a connector such as an anisotropic conductive film or a TCP (Tape Carrier Package), a module in which a printed wiring board is provided at the end of a TCP, and a module in which an IC (integrated circuit) is directly mounted on a light-emitting device by a COG (Chip On Glass) method. Furthermore, a lighting device or the like may include the light-emitting apparatus.

### Effect of the Invention

According to one embodiment of the present invention, a novel light-emitting device that is highly convenient, useful, or reliable can be provided. Alternatively, a novel light-emitting apparatus that is highly convenient, useful, or reliable can be provided. Alternatively, a novel display apparatus that is highly convenient, useful, or reliable can be provided. Alternatively, a novel electronic device that is highly convenient, useful, or reliable can be provided. Alternatively, a novel lighting device that is highly convenient, useful, or reliable can be provided. Alternatively, a novel light-emitting device, a novel light-emitting apparatus, a novel display apparatus, a novel electronic device, or a novel lighting device can be provided.

Note that the description of these effects does not preclude the existence of other effects. Note that one embodiment of the present invention does not need to have all the effects. Other effects will be apparent from the description of the specification, the drawings, the claims, and the like, and other effects can be derived from the description of the specification, the drawings, the claims, and the like.

### BRIEF DESCRIPTION OF THE DRAWINGS

FIG. 1A and FIG. 1B are diagrams illustrating a structure of a light-emitting device of an embodiment.

FIG. 2A and FIG. 2B are diagrams each illustrating a structure of a light-emitting device of an embodiment.

FIG. 3A and FIG. 3B are diagrams each illustrating a structure of a functional panel of an embodiment.

FIG. 4A and FIG. 4B are diagrams each illustrating a structure of a functional panel of an embodiment.

FIG. 5 is a diagram illustrating a structure of a functional panel of an embodiment.

FIG. 6A and FIG. 6B are conceptual diagrams of an active matrix light-emitting apparatus.

FIG. 7A and FIG. 7B are conceptual diagrams of an active matrix light-emitting apparatus.

FIG. 8 is a conceptual diagram of an active matrix light-emitting apparatus.

FIG. 9A and FIG. 9B are conceptual diagrams of a passive matrix light-emitting apparatus.

FIG. 10A and FIG. 10B are diagrams showing a lighting device.

FIG. 11A to FIG. 11D are diagrams showing electronic devices.

FIG. 12A to FIG. 12C are diagrams showing electronic devices.

FIG. 13 is a diagram showing a lighting device.

FIG. 14 is a diagram showing a lighting device.

FIG. 15 is a diagram showing in-vehicle display apparatuses and lighting devices.

FIG. 16A to FIG. 16C are diagrams showing an electronic device.

FIG. 17A and FIG. 17B are diagrams illustrating a structure of light-emitting devices of an example.

FIG. 18 is a diagram illustrating current density-luminance characteristics of light-emitting devices of an example.

FIG. 19 is a diagram illustrating luminance-current efficiency characteristics of light-emitting devices of an example.

FIG. 20 is a diagram illustrating voltage-luminance characteristics of light-emitting devices of an example.

FIG. 21 is a diagram illustrating voltage-current characteristics of light-emitting devices of an example.

FIG. 22 is a diagram illustrating luminance-blue index characteristics of light-emitting devices of an example.

FIG. 23 is a diagram illustrating emission spectra of light-emitting devices of an example.

FIG. 24 is a diagram illustrating temporal changes in normalized luminance of light-emitting devices of an example.

### MODE FOR CARRYING OUT THE INVENTION

A light-emitting device of one embodiment of the present invention includes a first electrode, a second electrode, a first unit, and a first layer. The first unit is held between the first electrode and the second electrode, and the first unit includes a second layer, a third layer, and a fourth layer. The second layer is held between the third layer and the fourth layer, and the second layer contains a light-emitting material. The fourth layer is held between the second layer and the second electrode, the fourth layer contains a first organic compound, the first organic compound has a π-electron deficient heteroaromatic ring skeleton and a π-electron rich heteroaromatic ring skeleton, and the first organic compound has a HOMO level higher than or equal to −6.0 eV and lower than or equal to −5.6 eV. The first layer is held between the first electrode and the first unit, and the first layer is in contact with the first electrode. The first layer contains a second organic compound and a third organic compound, the third organic compound has an electron-accepting property with respect to the second organic compound, and the first layer has a resistivity higher than or equal to 1×10⁴ [Ω·cm] and lower than or equal to 1×10⁷ [Ω·cm].

When the first organic compound has a diazine skeleton and a π-electron rich heteroaromatic ring skeleton, for example, electron transfer from the second electrode to the second layer can be facilitated. Moreover, when the first organic compound has a π-electron deficient heteroaromatic ring skeleton and a carbazole skeleton and has a HOMO level higher than or equal to −6.0 eV and lower than or equal to −5.6 eV, hole transfer from the second layer to the fourth layer can be facilitated. Accumulation of holes at the interface between the second layer and the fourth layer can be reduced and a change in the properties of the organic compound can be inhibited. Consequently, a novel light-emitting device that is highly convenient, useful, or reliable can be provided.

The high resistivity of the first layer is expected to have an effect of inhibiting a crosstalk. However, too high a resistivity prevents hole injection and does not lead to a light-emitting device with a favorable lifetime. Therefore, the resistivity of a material included in the first layer is preferably higher than or equal to 1×10⁴ [Ω·cm] and lower than or equal to 1×10⁷ [Ω·cm]. The light-emitting device has a favorable lifetime, and a light-emitting apparatus that includes the light-emitting device can have favorable display quality with suppressed crosstalk.

Embodiments are described in detail with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments. Note that in structures of the invention described below, the same portions or portions having similar functions are denoted by the same reference numerals in different drawings, and the description thereof is not repeated.

### Embodiment 1

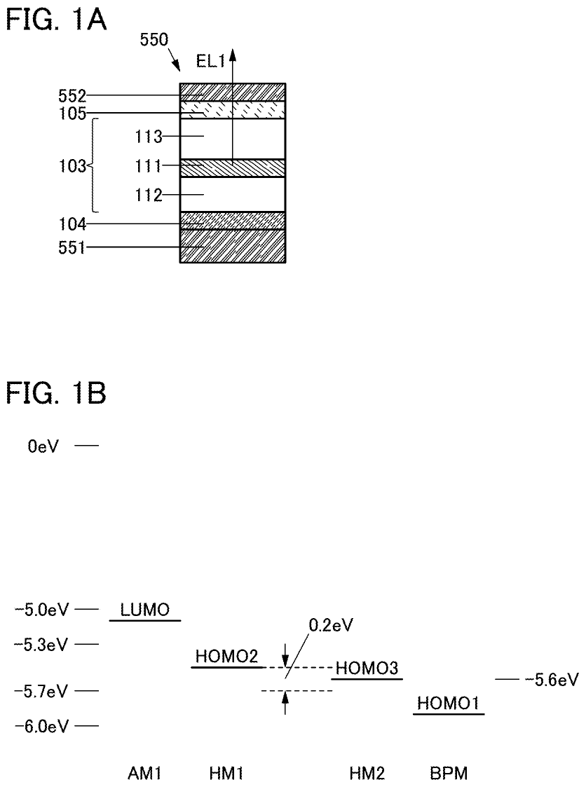

In this embodiment, a structure of a light-emitting device 550 of one embodiment of the present invention is described with reference to FIG. 1.

FIG. 1A is a cross-sectional view of the light-emitting device 550 of one embodiment of the present invention, and FIG. 1B is a diagram illustrating a structure of the light-emitting device 550 of one embodiment of the present invention.

### <Structure Example of Light-Emitting Device 550>

The light-emitting device described in this embodiment includes an electrode 551, an electrode 552, a unit 103, and a layer 104 (see FIG. 1A). The unit 103 is held between the electrode 551 and the electrode 552.

### <Structure Example of Electrode 551>

A conductive material can be used for the electrode 551, for example. Specifically, a single layer or a stacked layer of a metal, an alloy, or a film containing a conductive compound can be used for the electrode 551.

For example, a film that efficiently reflects light can be used for the electrode 551. Specifically, an alloy containing silver, copper, and the like, an alloy containing silver, palladium, and the like, or a metal film of aluminum or the like can be used for the electrode 551.

Alternatively, for example, a metal film that transmits part of light and reflects the other part of the light can be used as the electrode 551. Thus, a microcavity structure (microcavity) can be provided in the light-emitting device 150. Light of a predetermined wavelength can be extracted more efficiently than other light. Light with a narrow half width of a spectrum can be extracted. Light of a bright color can be extracted.

A film having a property of transmitting visible light can be used for the electrode 551, for example. Specifically, a single layer or a stacked layer of a metal film, an alloy film, a conductive oxide film, or the like that is thin enough to transmit light can be used for the electrode 551.

In particular, a material having a work function higher than or equal to 4.0 eV can be suitably used for the electrode 551.

For example, a conductive oxide containing indium can be used for the electrode 551. Specifically, indium oxide, indium oxide-tin oxide (abbreviation: ITO), indium oxide-tin oxide containing silicon or silicon oxide (abbreviation: ITSO), indium oxide-zinc oxide, indium oxide containing tungsten oxide and zinc oxide (IWZO), or the like can be used.

Furthermore, for example, a conductive oxide containing zinc can be used. Specifically, zinc oxide, zinc oxide to which gallium is added, zinc oxide to which aluminum is added, or the like can be used.

Furthermore, for example, gold (Au), platinum (Pt), nickel (Ni), tungsten (W), chromium (Cr), molybdenum (Mo), iron (Fe), cobalt (Co), copper (Cu), palladium (Pd), a nitride of a metal material (e.g., titanium nitride), or the like can be used. Alternatively, graphene can be used.

### <Structure Example of Unit 103>

The unit 103 includes a layer 111, a layer 112, and a layer 113 (see FIG. 1A). The unit 103 has a function of emitting light EL1.

For example, a layer selected from functional layers such as a light-emitting layer, a hole-transport layer, an electron-transport layer, and a carrier-blocking layer can be used in the unit 103. Moreover, a layer selected from functional layers such as a hole-injection layer, an electron-injection layer, an exciton-blocking layer, and a charge-generation layer can be used in the unit 103.

### [Structure Example 1 of Layer 111]

The layer 111 is held between the layer 112 and the layer 113, and the layer 111 contains a light-emitting material. A light-emitting material and a host material can be used for the layer 111. The layer 111 can be referred to as a light-emitting layer. The layer 111 is preferably provided in a region where holes and electrons are recombined. In that case, energy generated by recombination of carriers can be efficiently converted into light and emitted.

Furthermore, the layer 111 is preferably provided apart from a metal used for the electrode or the like. In that case, a quenching phenomenon caused by the metal used for the electrode or the like can be inhibited.

It is preferable that a distance from an electrode or the like having reflectivity to the layer 111 be adjusted and the layer 111 be placed in an appropriate position in accordance with an emission wavelength. Thus, the amplitude can be increased by utilizing an interference phenomenon between light reflected by the electrode or the like and light emitted from the layer 111. Light of a predetermined wavelength can be intensified and the spectrum of the light can be narrowed. In addition, bright light emission colors with high intensity can be obtained. In other words, the layer 111 is placed in an appropriate position, for example, between electrodes and the like, and thus a microcavity structure (microcavity) can be formed.

For example, a fluorescent substance, a phosphorescent substance, or a substance exhibiting thermally activated delayed fluorescence (TADF) (also referred to as a TADF material) can be used as the light-emitting material. Thus, energy generated by recombination of carriers can be released as the light EL1 from the light-emitting material (see FIG. 1A).

### [Fluorescent Substance]

A fluorescent substance can be used for the layer 111. For example, any of the following fluorescent substances can be used for the layer 111. Note that without being limited to the following ones, any of a variety of known fluorescent substances can be used for the layer 111.

Specifically, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl) biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPrn), N,N-bis(3-methylphenyl)-N,N′-bis[3-(9-phenyl-9H-fluoren-9-yl)phenyl]pyrene-1,6-diamine (abbreviation: 1,6mMemFLPAPrn), N,N-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl) perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N,N-(pyrene-1,6-diyl)bis[(6,N-diphenylbenzo[b]naphtho[1,2-d]furan)-8-amine] (abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), or 3,10-bis[N-(dibenzofuran-3-yl)-N-phenylamino]naphtho[2,3-b;6,7-b′]bisbenzofuran (abbreviation: 3,10FrA2Nbf (IV)-02).

Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, or high reliability.

In addition, it is possible to use, for example, N-[4-(9,10-diphenyl-2-anthryl)phenyl]-N,N′,N′-triphenyl-1,4-phenylenediamine (abbreviation: 2DPAPPA), N,N,N′,N′,N″,N″,N″,N″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1, l′-biphenyl-2-yl)-N-[4-(9H-carbazol-9-yl)phenyl]-N-phenylanthracene-2-amine (abbreviation: 2YGABPhA), N,N,9-triphenylanthracene-9-amine (abbreviation: DPhAPhA), coumarin 545T, N,N′-diphenylquinacridone (abbreviation: DPQd), rubrene, or 5,12-bis(1,1′-biphenyl-4-yl)-6,11-diphenyltetracene (abbreviation: BPT).

Furthermore, it is possible to use, for example, 2-(2-{2-[4-(dimethylamino)phenyl]ethenyl}-6-methyl-4H-pyran-4-ylidene) propanedinitrile (abbreviation: DCM1), 2-{2-methyl-6-[2-(2,3,6,7-tetrahydro-1H,5H-benzo[ij]quinolizin-9-yl) ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl) acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl) ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl) ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene) propanedinitrile (abbreviation: BisDCM), or 2-{2,6-bis[2-(8-methoxy-1,1,7,7-tetramethyl-2,3,6,7-tetrahydro-11,5H-benzo[ij]quinolizin-9-yl) ethenyl]-4H-pyran-4-ylidene}propanedinitrile (abbreviation: BisDCJTM).

### [Phosphorescent Substance]

A phosphorescent substance can be used for the layer 111. For example, any of the following phosphorescent substances can be used for the layer 111. Note that without being limited to the following ones, any of a variety of known phosphorescent substances can be used for the layer 111.

For the layer 111, it is possible to use, for example, an organometallic iridium complex having a 4H-triazole skeleton, an organometallic iridium complex having a 1H-triazole skeleton, an organometallic iridium complex having an imidazole skeleton, an organometallic iridium complex having a phenylpyridine derivative with an electron-withdrawing group as a ligand, an organometallic iridium complex having a pyrimidine skeleton, an organometallic iridium complex having a pyrazine skeleton, an organometallic iridium complex having a pyridine skeleton, a rare earth metal complex, or a platinum complex.

### [Phosphorescent Substance (Blue)]

As an organometallic iridium complex having a 4H-triazole skeleton or the like, tris {2-[5-(2-methylphenyl)-4-(2,6-dimethylphenyl)-4/H-1,2,4-triazol-3-yl-κN²]phenyl-κC²}iridium(III) (abbreviation: [Ir(mpptz-dmp)₃]), tris(5-methyl-3,4-diphenyl-4H-1,2,4-triazolato) iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), or the like can be used.

As an organometallic iridium complex having a 1H-triazole skeleton or the like, tris[3-methyl-1-(2-methylphenyl)-5-phenyl-1H-1,2,4-triazolato]iridium(III) (abbreviation: [Ir(Mptz1-mp)₃]), tris(1-methyl-5-phenyl-3-propyl-1H-1,2,4-triazolato]iridium(III) (abbreviation: [Ir(Prptz1-Me)₃]), or the like can be used.

As an organometallic iridium complex having an imidazole skeleton or the like, fac-tris[1-(2,6-diisopropylphenyl)-2-phenyl-1H-imidazole]iridium(III) (abbreviation: [Ir(iPrpmi)₃]), tris[3-(2,6-dimethylphenyl)-7-methylimidazo[1,2-f]phenanthridinato]iridium(III) (abbreviation: [Ir(dmpimpt-Me)₃]), or the like can be used.

As an organometallic iridium complex having a phenylpyridine derivative with an electron-withdrawing group as a ligand, or the like, bis[2-(4′,6′-difluorophenyl)pyridinato-N,C²′]iridium(III) tetrakis(1-pyrazolyl)borate (abbreviation: FIr6), bis[2-(4′,6′-difluorophenyl)pyridinato-N,C²′]iridium(III) picolinate (abbreviation: FIrpic), bis{2-[3′,5′-bis(trifluoromethyl)phenyl]pyridinato-N,C²′}iridium(III) picolinate (abbreviation: [Ir(CF₃ppy)₂(pic)]), bis[2-(4′,6′-difluorophenyl)pyridinato-N,C²′]iridium(III) acetylacetonate (abbreviation: FIracac), or the like can be used.

Note that these are compounds exhibiting blue phosphorescence and are compounds having an emission wavelength peak at 440 nm to 520 nm.

### [Phosphorescent Substance (Green)]

As an organometallic iridium complex having a pyrimidine skeleton or the like, it is possible to use, for example, tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), or (acetylacetonato)bis(4,6-diphenylpyrimidinato)iridium(III) (abbreviation: [Ir(dppm)₂(acac)]).

As an organometallic iridium complex having a pyrazine skeleton or the like, (acetylacetonato)bis(3,5-dimethyl-2-phenylpyrazinato)iridium(III) (abbreviation: [Ir(mppr-Me)₂(acac)]), (acetylacetonato)bis(5-isopropyl-3-methyl-2-phenylpyrazinato)iridium(III) (abbreviation: [Ir(mppr-iPr)₂(acac)]), or the like can be used.

As an organometallic iridium complex having a pyridine skeleton or the like, it is possible to use, for example, tris(2-phenylpyridinato-N,C²′)iridium(III) (abbreviation: [Ir(ppy)₃]), bis(2-phenylpyridinato-N,C²′)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²′)iridium(III) (abbreviation: [Ir(pq)₃]), bis(2-phenylquinolinato-N,C²′)iridium(III) acetylacetonate (abbreviation: [Ir(pq)₂(acac)]), [2-d3-methyl-8-(2-pyridinyl-κN)benzofuro[2,3-b]pyridine-κC]bis[2-(5-d3-methyl-2-pyridinyl-κN²)phenyl-κC′]iridium(III) (abbreviation: [Ir(5mppy-d3)₂(mbfpypy-d3)]), or [2-d3-methyl-(2-pyridinyl-κN)benzofuro[2,3-b]pyridine-κC]bis[2-(2-pyridinyl-κN)phenyl-κN]iridium(III) (abbreviation: [Ir(ppy)₂(mbfpypy-d3)]).

An example of a rare earth metal complex is tris(acetylacetonato)(monophenanthroline)terbium(III) (abbreviation: [Tb(acac)₃(Phen)]).

Note that these are compounds mainly exhibiting green phosphorescence and have an emission wavelength peak at 500 nm to 600 nm. An organometallic iridium complex having a pyrimidine skeleton excels particularly in reliability or emission efficiency.

### [Phosphorescent Substance (Red)]

As an organometallic iridium complex having a pyrimidine skeleton or the like, (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), bis[4,6-di(naphthalen-1-yl)pyrimidinato](dipivaloylmethanato)iridium(III) (abbreviation: [Ir(d1npm)₂(dpm)]), or the like can be used.

As an organometallic iridium complex having a pyrazine skeleton or the like, (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), (acetylacetonato)bis[2,3-bis(4-fluorophenyl)quinoxalinato]iridium(III) (abbreviation: [Ir(Fdpq)₂(acac)]), or the like can be used.

As an organometallic iridium complex having a pyridine skeleton or the like, tris(1-phenylisoquinolinato-N,C²′)iridium(III) (abbreviation: [Ir(piq)₃]), bis(1-phenylisoquinolinato-N,C²′)iridium(III) acetylacetonate (abbreviation: [Ir(piq)₂(acac)]), or the like can be used.

As a rare earth metal complex or the like, tris(1,3-diphenyl-1,3-propanedionato)(monophenanthroline)europium(III) (abbreviation: [Eu(DBM)₃(Phen)]), tris[1-(2-thenoyl)-3,3,3-trifluoroacetonato](monophenanthroline)europium(III) (abbreviation: [Eu(TTA)₃(Phen)]), or the like can be used.

As a platinum complex or the like, 2,3,7,8,12,13,17,18-octaethyl-21H,23H-porphyrin platinum (II) (abbreviation: PtOEP) or the like can be used.

Note that these are compounds exhibiting red phosphorescence and have an emission peak at 600 nm to 700 nm. Furthermore, from the organometallic iridium complex having a pyrazine skeleton, red light emission with chromaticity favorably used for display apparatuses can be obtained.

### [Substance Exhibiting Thermally Activated Delayed Fluorescence (TADF)]

A TADF material can be used for the layer 111. For example, any of the TADF materials given below can be used as the light-emitting material. Note that without being limited thereto, any of a variety of known TADF materials can be used as the light-emitting material.

In the TADF material, the difference between the S1 level and the T1 level is small, and reverse intersystem crossing (upconversion) from the triplet excited state into the singlet excited state can be achieved by a little thermal energy. Thus, the singlet excited state can be efficiently generated from the triplet excited state. In addition, the triplet excitation energy can be converted into light.

An exciplex whose excited state is formed of two kinds of substances has an extremely small difference between the S1 level and the T1 level and functions as a TADF material capable of converting triplet excitation energy into singlet excitation energy.

A phosphorescence spectrum observed at a low temperature (e.g., 77 K to 10 K) is used for an index of the T1 level. When the level of energy with a wavelength at which the horizontal axis intersects the line obtained by extrapolating a tangent to the fluorescence spectrum at a tail on the short wavelength side is the S1 level and the level of energy with a wavelength of the line obtained by extrapolating a tangent to the phosphorescence spectrum at a tail on the short wavelength side is the T1 level, the difference between the S1 level and the T1 level of the TADF material is preferably smaller than or equal to 0.3 eV, further preferably smaller than or equal to 0.2 eV.

When a TADF material is used as the light-emitting substance, the S1 level of the host material is preferably higher than that of the TADF material. In addition, the T1 level of the host material is preferably higher than that of the TADF material.

Examples of the TADF material include a fullerene, a derivative thereof, an acridine, a derivative thereof, and an eosin derivative. Furthermore, porphyrin containing a metal such as magnesium (Mg), zinc (Zn), cadmium (Cd), tin (Sn), platinum (Pt), indium (In), or palladium (Pd) can be also used for the TADF material.

Specifically, any of the following materials whose structural formulae are shown below can be used: a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), an octaethylporphyrin-platinum chloride complex (PtCl₂OEP), and the like.

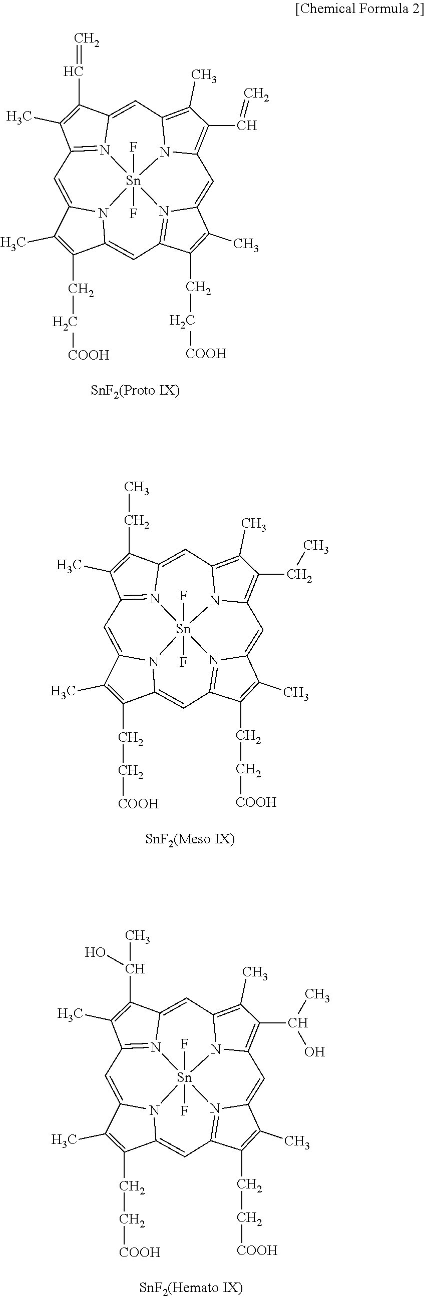

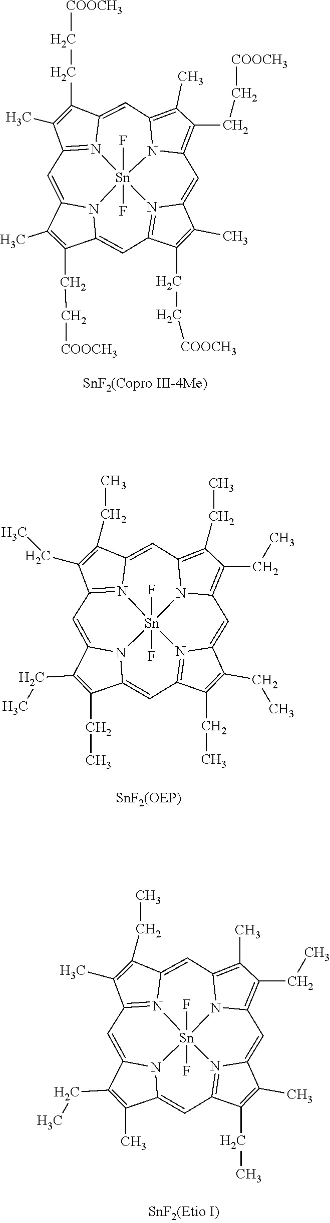

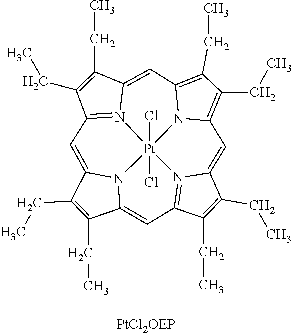

Furthermore, a heterocyclic compound including one or both of a π-electron rich heteroaromatic ring and a π-electron deficient heteroaromatic ring can be used, for example, for the TADF material.

Specifically, any of the following materials whose structural formulae are shown below can be used: 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), and the like.

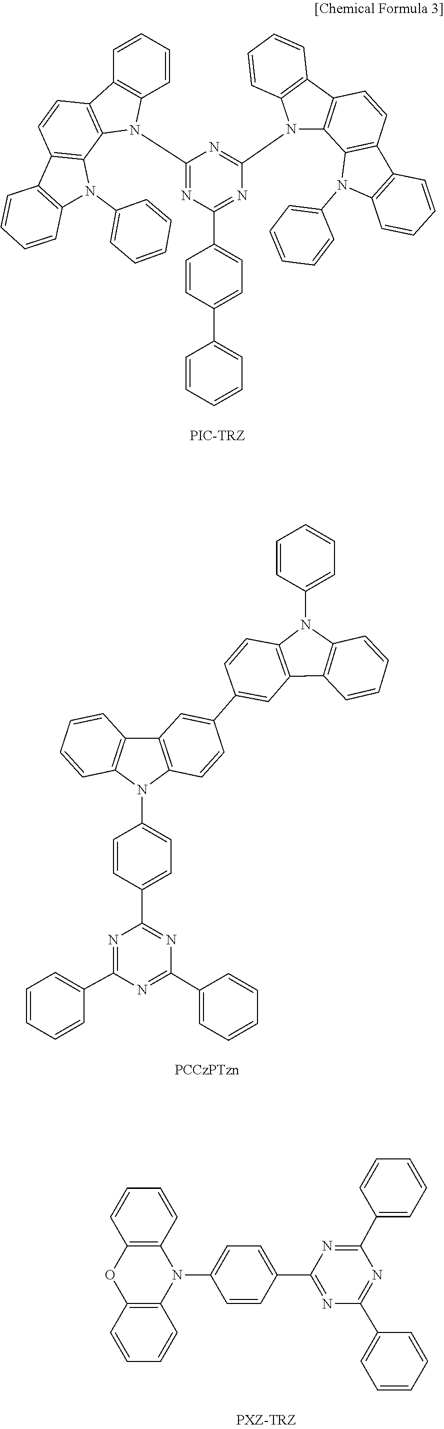 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3ccc(N4c5ccccc5Oc5ccccc54)cc3)n2)cc1]

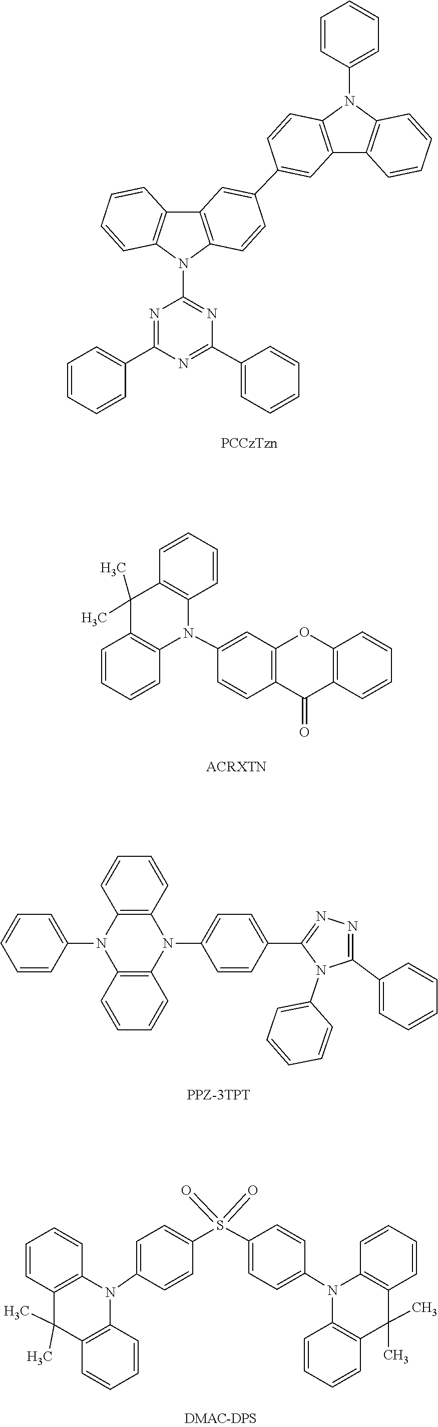

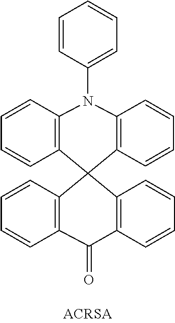 [SMILES: O=C1c2ccccc2C2(c3ccccc31)c1ccccc1N(c1ccccc1)c1ccccc12]

Such a heterocyclic compound is preferable because of having excellent electron-transport property and hole-transport property owing to a π-electron rich heteroaromatic ring and a π-electron deficient heteroaromatic ring. Among skeletons having the π-electron deficient heteroaromatic ring, in particular, a pyridine skeleton, a diazine skeleton (a pyrimidine skeleton, a pyrazine skeleton, and a pyridazine skeleton), and a triazine skeleton are preferable because they are stable. In particular, a benzofuropyrimidine skeleton, a benzothienopyrimidine skeleton, a benzofuropyrazine skeleton, and a benzothienopyrazine skeleton are preferable because of their high electron-accepting properties and reliability.

Among skeletons having the π-electron rich heteroaromatic ring, an acridine skeleton, a phenoxazine skeleton, a phenothiazine skeleton, a furan skeleton, a thiophene skeleton, and a pyrrole skeleton are stable; therefore, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable.

Note that a substance in which the π-electron rich heteroaromatic ring is directly bonded to the π-electron deficient heteroaromatic ring is particularly preferable because the electron-donating property of the π-electron rich heteroaromatic ring and the electron-accepting property of the π-electron deficient heteroaromatic ring are both improved, the energy difference between the S1 level and the T1 level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used.

As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a nitrile group or a cyano group such as benzonitrile or cyanobenzene, a carbonyl skeleton such as benzophenone, a phosphine oxide skeleton, a sulfone skeleton, or the like can be used.

As described above, a π-electron deficient skeleton and a π-electron rich skeleton can be used instead of at least one of the π-electron deficient heteroaromatic ring and the π-electron rich heteroaromatic ring.

### Structure Example 2 of Layer 111

A material having a carrier-transport property can be used as the host material. For example, a material having a hole-transport property, a material having an electron-transport property, a substance exhibiting thermally activated delayed fluorescence TADF, a material having an anthracene skeleton, or a mixed material can be used as the host material. A material having a wider band gap than the light-emitting material contained in the layer 111 is preferably used as the host material. In that case, energy transfer from excitons generated in the layer 111 to the host material can be inhibited.

### [Material Having Hole-Transport Property]

A material having a hole mobility higher than or equal to 1×10⁻⁶ cm²/Vs can be suitably used as the material having a hole-transport property.

As the material having a hole-transport property, an amine compound or an organic compound having a π-electron rich heteroaromatic ring skeleton can be used, for example. Specifically, a compound having an aromatic amine skeleton, a compound having a carbazole skeleton, a compound having a thiophene skeleton, a compound having a furan skeleton, or the like can be used. In particular, a compound having an aromatic amine skeleton and a compound having a carbazole skeleton are preferable because these have favorable reliability, have high hole-transport properties, and contribute to a reduction in driving voltage.

The following are examples that can be used as the compound having an aromatic amine skeleton: 4,4′-bis[N-(1-naphthyl)-N-phenylamino]biphenyl (abbreviation: NPB), N,N-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]spiro-9,9′-bifluoren-2-amine (abbreviation: PCBASF).

As the compound having a carbazole skeleton, for example, 1,3-bis(N-carbazolyl)benzene (abbreviation: mCP), 4,4′-di(N-carbazolyl) biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), or the like can be used.

As the compound having a thiophene skeleton, for example, 4,4′,4″-(benzene-1,3,5-triyl)tri (dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), or the like can be used.

As the compound having a furan skeleton, for example, 4,4′,4″-(benzene-1,3,5-triyl)tri (dibenzofuran) (abbreviation: DBF3P-II), 4-{3-[3-(9-phenyl-9H-fluoren-9-yl)phenyl]phenyl}dibenzofuran (abbreviation: mmDBFFLBi-II), or the like can be used.

### [Material Having Electron-Transport Property]

For example, a metal complex or an organic compound having a π-electron deficient heteroaromatic ring skeleton can be used as the material having an electron-transport property.

As the metal complex, bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), bis[2-(2-benzothiazolyl)phenolato]zinc(II) (abbreviation: ZnBTZ), or the like can be used, for example.

As the organic compound having a π-electron deficient heteroaromatic ring skeleton, a heterocyclic compound having a polyazole skeleton, a heterocyclic compound having a diazine skeleton, a heterocyclic compound having a pyridine skeleton, a heterocyclic compound having a triazine skeleton, or the like can be used, for example. In particular, the heterocyclic compound having a diazine skeleton or the heterocyclic compound having a pyridine skeleton has favorable reliability and thus is preferable. Furthermore, the heterocyclic compound having a diazine (pyrimidine or pyrazine) skeleton has a high electron-transport property and can contribute to a reduction in driving voltage.

As the heterocyclic compound having a polyazole skeleton, 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or the like can be used, for example.

As the heterocyclic compound having a diazine skeleton, 2-[3-(dibenzothiophen-4-yl)phenyl]dibenzo[f,h]quinoxaline (abbreviation: 2mDBTPDBq-II), 2-[3′-(dibenzothiophen-4-yl) biphenyl-3-yl]dibenzo[f,h]quinoxaline (abbreviation: 2mDBTBPDBq-II), 2-[3′-(9H-carbazol-9-yl) biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,8-bis[3-(dibenzothiophen-4-yl)phenyl]benzo[h]quinazoline (abbreviation: 4,8mDBtP2Bqn), or the like can be used, for example.

As the heterocyclic compound having a pyridine skeleton, 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), or the like can be used, for example.

As the heterocyclic compound having a triazine skeleton, 2-[3′-(9,9-dimethyl-9H-fluoren-2-yl) biphenyl-3-yl]-4,6-diphenyl-1,3,5-triazine (abbreviation: mFBPTzn), 2-[(1,1′-biphenyl)-4-yl]-4-phenyl-6-[9,9′-spirobi (9H-fluoren)-2-yl]-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), or 2-{3-[3-(benzo[b]naphtho[1,2-d]furan-6-yl)phenyl]phenyl}-4,6-diphenyl-1,3,5-triazine (abbreviation: mBnfBPTzn-02) can be used, for example.

### [Material Having Anthracene Skeleton]

An organic compound having an anthracene skeleton can be used as the host material. In particular, when a fluorescent substance is used as the light-emitting substance, an organic compound having an anthracene skeleton is preferably used. In that case, a light-emitting device with high emission efficiency and high durability can be obtained.

As the organic compound having an anthracene skeleton, an organic compound having a diphenylanthracene skeleton, in particular, a 9,10-diphenylanthracene skeleton is chemically stable and thus is preferable. The host material preferably has a carbazole skeleton, in which case the hole-injection and hole-transport properties are improved. In particular, the host material preferably has a dibenzocarbazole skeleton, in which case the HOMO level thereof is shallower than that of carbazole by approximately 0.1 eV, so that holes enter the host material easily, the hole-transport property is improved, and the heat resistance is increased. Note that in terms of the hole-injection and hole-transport properties, a benzofluorene skeleton or a dibenzofluorene skeleton may be used instead of a carbazole skeleton.

Thus, a substance having both a 9,10-diphenylanthracene skeleton and a carbazole skeleton, a substance having both a 9,10-diphenylanthracene skeleton and a benzocarbazole skeleton, or a substance having both a 9,10-diphenylanthracene skeleton and a dibenzocarbazole skeleton is preferable as the host material.

For example, it is possible to use 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl) biphenyl-4′-yl}anthracene (abbreviation: FLPPA), 9-(1-naphthyl)-10-[4-(2-naphthyl)phenyl]anthracene (abbreviation: αN-BNPAnth), 9-phenyl-3-[4-(10-phenyl-9-anthryl)phenyl]-9H-carbazole (abbreviation: PCzPA), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), or 3-[4-(1-naphthyl)-phenyl]-9-phenyl-9H-carbazole (abbreviation: PCPN).

In particular, CzPA, cgDBCzPA, 2mBnfPPA, and PCzPA have excellent characteristics.

### [Substance Exhibiting Thermally Activated Delayed Fluorescence (TADF)]

A TADF material can be used as the host material. When the TADF material is used as the host material, triplet excitation energy generated in the TADF material can be converted into singlet excitation energy by reverse intersystem crossing. Moreover, excitation energy can be transferred to the light-emitting substance. In other words, the TADF material functions as an energy donor, and the light-emitting substance functions as an energy acceptor. Thus, the emission efficiency of the light-emitting device can be increased.

This is very effective in the case where the light-emitting substance is a fluorescent substance. In that case, the S1 level of the TADF material is preferably higher than that of the fluorescent substance in order that high emission efficiency can be achieved. Furthermore, the T1 level of the TADF material is preferably higher than the S1 level of the fluorescent substance. Therefore, the T1 level of the TADF material is preferably higher than that of the fluorescent substance.

It is also preferable to use a TADF material that emits light whose wavelength overlaps with the wavelength of a lowest-energy-side absorption band of the fluorescent substance, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protecting group around a luminophore (a skeleton which causes light emission) of the fluorescent substance. As the protecting group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protecting groups. The substituents having no π bond are poor in carrier-transport performance, whereby the TADF material and the luminophore of the fluorescent substance can be made away from each other with little influence on carrier transport or carrier recombination.

Here, the luminophore refers to an atomic group (skeleton) that causes light emission in a fluorescent substance. The luminophore is preferably a skeleton having a π bond, further preferably includes an aromatic ring, still further preferably includes a condensed aromatic ring or a condensed heteroaromatic ring.

Examples of the condensed aromatic ring or the condensed heteroaromatic ring include a phenanthrene skeleton, a stilbene skeleton, an acridone skeleton, a phenoxazine skeleton, and a phenothiazine skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

For example, the TADF material that can be used as the light-emitting material can be used as the host material.

### [Structure Example 1 of Mixed Material]

A material in which a plurality of kinds of substances are mixed can be used as the host material. For example, a material that contains an electron-transport material and a hole-transport material can be used as the mixed material. The weight ratio between the hole-transport material and the electron-transport material contained in the mixed material may be (the hole-transport material/the electron-transport material)=(1/19) or more and (19/1) or less. Accordingly, the carrier-transport property of the layer 111 can be easily adjusted. In addition, a recombination region can be controlled easily.

### [Structure Example 2 of Mixed Material]

A material mixed with a phosphorescent substance can be used as the host material. When a fluorescent substance is used as the light-emitting substance, a phosphorescent substance can be used as an energy donor for supplying excitation energy to the fluorescent substance.

### [Structure Example 3 of Mixed Material]

A mixed material containing a material to form an exciplex can be used as the host material. For example, a material forming an exciplex whose emission spectrum overlaps with the wavelength of the absorption band on the lowest energy side of the light-emitting substance can be used as the host material. This enables smooth energy transfer and improves emission efficiency. Alternatively, the driving voltage can be reduced. With such a structure, light emission can be efficiently obtained by ExTET (Exciplex-Triplet Energy Transfer), which is energy transfer from an exciplex to a light-emitting substance (a phosphorescent material).

A phosphorescent substance can be used as at least one of the materials forming an exciplex. Accordingly, reverse intersystem crossing can be used. Alternatively, triplet excitation energy can be efficiently converted into singlet excitation energy.

A combination of materials forming an exciplex is preferably such that the HOMO level of a material having a hole-transport property is higher than or equal to the HOMO level of a material having an electron-transport property. Alternatively, the LUMO level of the material having a hole-transport property is preferably higher than or equal to the LUMO level of the material having an electron-transport property. In that case, an exciplex can be efficiently formed. Note that the LUMO levels and the HOMO levels of the materials can be derived from the electrochemical characteristics (the reduction potentials and the oxidation potentials). Specifically, the reduction potentials and the oxidation potentials can be measured by cyclic voltammetry (CV).

The formation of an exciplex can be confirmed by a phenomenon in which the emission spectrum of a mixed film in which the material having a hole-transport property and the material having an electron-transport property are mixed is shifted to a longer wavelength than the emission spectrum of each of the materials (or has another peak on the longer wavelength side) observed in comparison of the emission spectrum of the material having a hole-transport property, the emission spectrum of the material having an electron-transport property, and the emission spectrum of the mixed film of these materials, for example. Alternatively, the formation of an exciplex can be confirmed by a difference in transient response, such as a phenomenon in which the transient photoluminescence (PL) lifetime of the mixed film has longer lifetime components or has a larger proportion of delayed components than that of each of the materials, observed in comparison of transient PL of the material having a hole-transport property, the transient PL of the material having an electron-transport property, and the transient PL of the mixed film of these materials. The transient PL can be rephrased as transient electroluminescence (EL). That is, the formation of an exciplex can also be confirmed by a difference in transient response observed in comparison of the transient EL of the material having a hole-transport property, the transient EL of the material having an electron-transport property, and the transient EL of the mixed film of these materials.

### <<Structure Example of Layer 113>>

The layer 113 is held between the layer 111 and the electrode 552 and has a single-layer structure or a stacked-layer structure. The layer 113 contains an organic compound BPM. For example, a material having an electron-transport property can be used for the layer 113. The layer 113 can be referred to as an electron-transport layer. A material having a wider band gap than the light-emitting material contained in the layer 111 is preferably used for the layer 113. In that case, energy transfer from excitons generated in the layer 111 to the layer 113 can be inhibited.

### [Example 1 of Organic Compound BPM]

The organic compound BPM has a π-electron deficient heteroaromatic ring skeleton and a π-electron rich heteroaromatic ring skeleton.

The organic compound BPM has a HOMO level HOMO1. The HOMO level HOMO1 is higher than or equal to −6.0 eV and lower than or equal to −5.6 eV (see FIG. 1B).

Examples of the π-electron rich heteroaromatic ring skeleton include a carbazole skeleton, an acridine skeleton, a phenoxazine skeleton, a phenothiazine skeleton, a furan skeleton, a thiophene skeleton, and a pyrrole skeleton. Specifically, when the organic compound BPM has a carbazole skeleton, the HOMO level HOMO1 of the organic compound BPM easily falls within a favorable range. Moreover, the HOMO level HOMO1 of the organic compound BPM can be easily controlled.

Examples of the π-electron deficient heteroaromatic ring skeleton include a pyridine skeleton, a diazine skeleton (a pyrimidine skeleton, a pyrazine skeleton, or a pyridazine skeleton), and a triazine skeleton.

### [Example 2 of Organic Compound BPM]

As the organic compound BPM having a π-electron deficient heteroaromatic ring skeleton and a carbazole skeleton, for example, 3,5-bis[3-(9H-carbazol-9-yl)phenyl]pyridine (abbreviation: 35DCzPPy), 2-[3′-(9H-carbazol-9-yl) biphenyl-3-yl]dibenzo[f,h]quinoxaline (abbreviation: 2mCzBPDBq), 2-[4′-(9-phenyl-9H-carbazol-3-yl)-3, l′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 6-(1,1′-biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(1,1′-biphenyl-4-yl)pyrimidine (abbreviation: 6BP-4Cz2PPm), 7-[4-(9-phenyl-9H-carbazol-2-yl) quinazolin-2-yl]-7H-dibenzo[c,g]carbazole (abbreviation: PC-cgDBCzQz), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 11-(4-[1,1′-diphenyl]-4-yl-6-phenyl-1,3,5-triazin-2-yl)-11,12-dihydro-12-phenyl-indolo[2,3-a]carbazole (abbreviation: BP-Icz(II)Tzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 3-[9-(4,6-diphenyl-1,3,5-triazin-2-yl)-2-dibenzofuranyl]-9-phenyl-9H-carbazole (abbreviation: PCDBfTzn), 2-{3-[3-(N-phenyl-9H-carbazol-3-yl)-9H-carbazol-9-yl]phenyl}dibenzo[f,h]quinoxaline (abbreviation: 2mPCCzPDBq), or the like can be used.

### [Example 3 of Organic Compound BPM]

The organic compound BPM is represented by General Formula (G1) below.

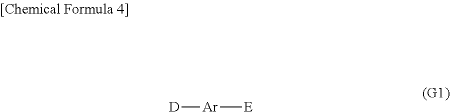

In General Formula (G1) above, D represents a substituted or unsubstituted quinoxalinyl group.

The substituted or unsubstituted quinoxalinyl group can be represented by General Formula (D-1) below, for example. One of R¹ to R¹⁰ is Ar and the others are hydrogen, a hydrocarbon group having 1 to 10 carbon atoms, an alicyclic hydrocarbon group having 3 to 10 carbon atoms, or a substituted or unsubstituted aromatic hydrocarbon group having 6 to 14 carbon atoms. As a substituent included in the aromatic hydrocarbon group, for example, an alkyl group having 1 to 4 carbon atoms, a substituted or unsubstituted cycloalkyl group having 3 to 10 carbon atoms, a substituted or unsubstituted aromatic hydrocarbon group having 6 to 30 carbon atoms, a substituted or unsubstituted heteroaromatic hydrocarbon group having 2 to 30 carbon atoms, or the like can be used.

More specifically, a methyl group, an ethyl group, a propyl group, an isopropyl group, a butyl group, an isobutyl group, a tert-butyl group, an n-hexyl group, or the like can be used as the substituent. Alternatively, a cyclopropyl group, a cyclobutyl group, a cyclopentyl group, a cyclohexyl group, an adamantyl group, or the like can be used as the substituent. Alternatively, a phenyl group, a naphthyl group, a biphenyl group, a fluorenyl group, a spirofluorenyl group, or the like can be used as the substituent. Alternatively, a pyridine ring, a diazine ring (a pyrimidine ring, a pyrazine ring, or a pyridazine ring), a triazine ring, a quinoline ring, a quinoxaline ring, a quinazoline ring, a benzoquinazoline ring, a phenanthroline ring, an azafluoranthene ring, an imidazole ring, an oxazole ring, an oxadiazole ring, a triazole ring, or the like can be used as the substituent.

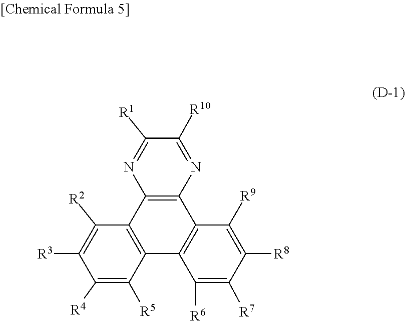

In General Formula (G1) above, E represents a substituted or unsubstituted carbazolyl group.

The substituted or unsubstituted carbazolyl group can be represented by General Formula (E-1) below, for example. One of R²¹ to R²⁹ is Ar and the others are hydrogen, a hydrocarbon group having 1 to 10 carbon atoms, an alicyclic hydrocarbon group having 3 to 10 carbon atoms, or a substituted or unsubstituted aromatic hydrocarbon group having 6 to 14 carbon atoms. As a substituent included in the aromatic hydrocarbon group, for example, an alkyl group having 1 to 4 carbon atoms, a substituted or unsubstituted cycloalkyl group having 3 to 10 carbon atoms, a substituted or unsubstituted aromatic hydrocarbon group having 6 to 30 carbon atoms, a substituted or unsubstituted heteroaromatic hydrocarbon group having 2 to 30 carbon atoms, or the like can be used. More specifically, any of the substituents that have been described can be used as the substituent.

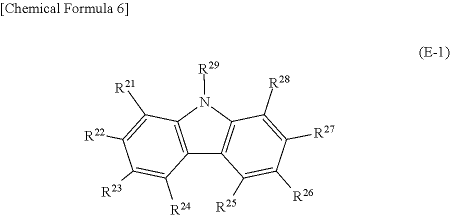

In General Formula (G1) above, Ar represents a substituted or unsubstituted arylene group, and the aromatic hydrocarbon group has 6 to 13 carbon atoms forming a ring.

The substituted or unsubstituted arylene group can be represented by any of General Formulae (Ar-1) to (Ar-14) below, for example. Note that Ar may have a substituent with a π-electron deficient heteroaromatic ring skeleton or a substituent with a π-electron rich heteroaromatic ring skeleton. In other words, a substituent with a π-electron deficient heteroaromatic ring skeleton or a π-electron rich heteroaromatic ring skeleton may be included in addition to D or E in General Formula (G1) above. Therefore, for example, a plurality of quinoxalinyl groups may be bonded to Ar; for another example, a plurality of carbazolyl groups may be bonded to Ar. As a substituent included in the arylene group, for example, an alkyl group having 1 to 4 carbon atoms, a substituted or unsubstituted cycloalkyl group having 3 to 10 carbon atoms, a substituted or unsubstituted aromatic hydrocarbon group having 6 to 30 carbon atoms, a substituted or unsubstituted heteroaromatic hydrocarbon group having 2 to 30 carbon atoms, or the like can be used. More specifically, any of the substituents that have been described can be used as the substituent.

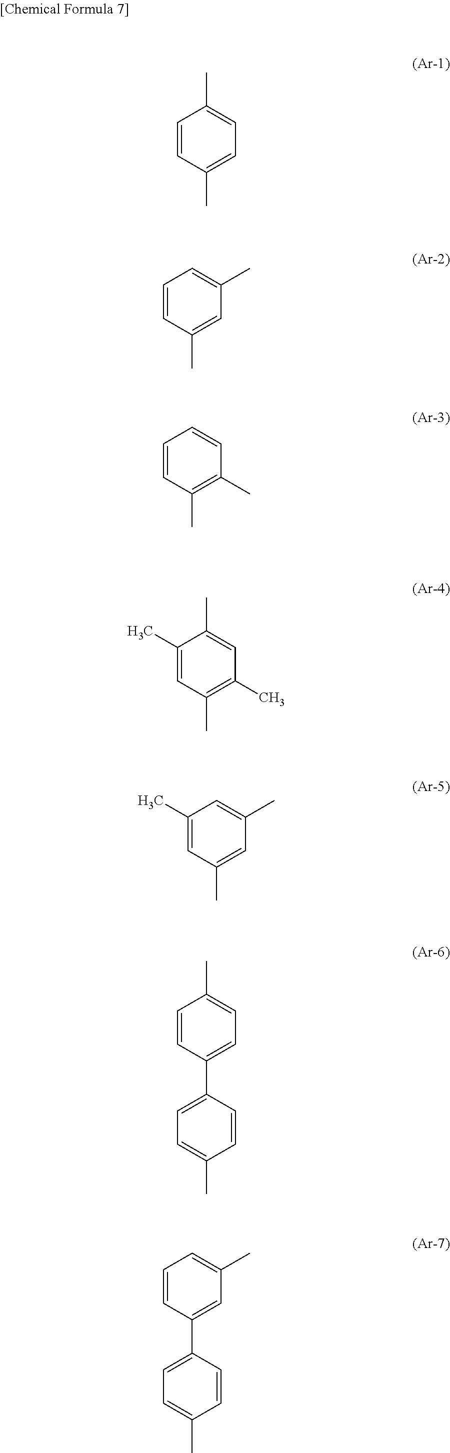

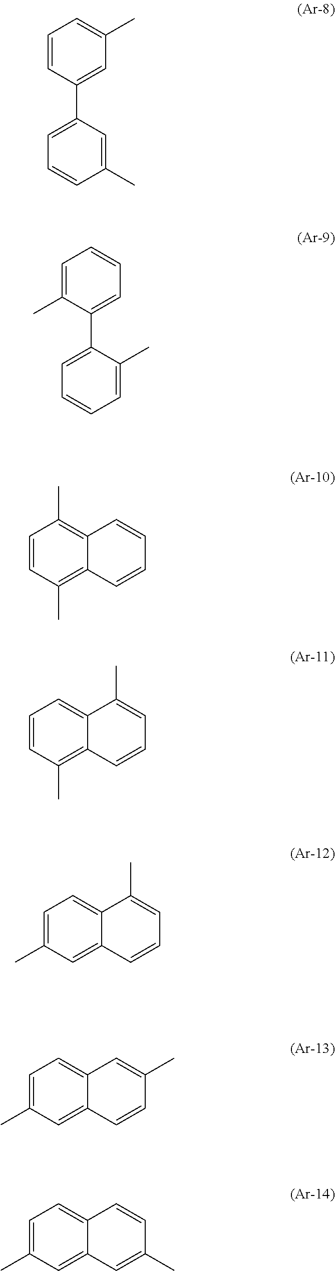

### [Example 4 of Organic Compound BPM]

Specifically, any of the organic compounds shown below, including 2-[4′-(9-phenyl-9H-carbazol-3-yl)-3,l′-biphenyl-1-yl]dibenzo[f,h]quinoxaline (abbreviation: 2mpPCBPDBq) and 3-[3,5-di(carbazol-9-yl)phenyl]phenanthro[9,10-b]pyrazine (abbreviation: 2Cz2PDBq), can be suitably used as the organic compound BPM.

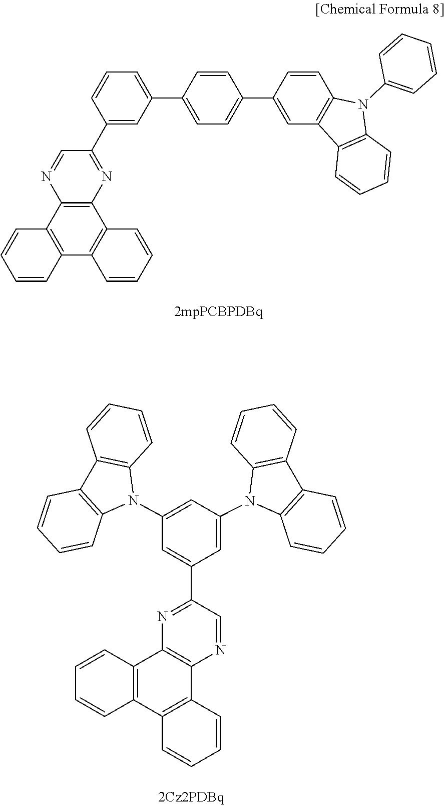 [SMILES: c1ccc(-n2c3ccccc3c3cc(-c4ccc(-c5cccc(-c6cnc7c8ccccc8c8ccccc8c7n6)c5)cc4)ccc32)cc1 ; c1ccc2c(c1)c1ccccc1c1nc(-c3cc(-n4c5ccccc5c5ccccc54)cc(-n4c5ccccc5c5ccccc54)c3)cnc21]

When the organic compound BPM has a diazine skeleton and a π-electron rich heteroaromatic ring skeleton, electron transfer from the electrode 552 to the layer 111 can be facilitated. Moreover, when the organic compound BPM has a π-electron deficient heteroaromatic ring skeleton and a carbazole skeleton and has the HOMO level HOMO1 higher than or equal to −6.0 eV and lower than or equal to −5.6 eV, hole transfer from the layer 111 to the layer 113 can be facilitated. Accumulation of holes at the interface between the layer 111 and the layer 113 can be reduced and a change in the properties of the organic compound can be inhibited. Consequently, a novel light-emitting device that is highly convenient, useful, or reliable can be provided.

### <<Structure Example 1 of Layer 104>>

The layer 104 is held between the electrode 551 and the unit 103 and is in contact with the electrode 551.

A material having a hole-injection property can be used for the layer 104. The layer 104 can be referred to as a hole-injection layer. The layer 104 contains, for example, an organic compound HM1 and an organic compound AM1.

The organic compound AM1 has an electron-accepting property with respect to the organic compound HM1. This can facilitate injection of holes from the electrode 551, for example. Alternatively, the driving voltage of the light-emitting device can be lowered.

An organic compound and an inorganic compound can be used as the substance having an electron-accepting property. The substance having an electron-accepting property can extract electrons from an adjacent hole-transport layer or an adjacent material having a hole-transport property by the application of an electric field.

For example, a compound having an electron-withdrawing group (a halogen group or a cyano group) can be used as the substance having an electron-accepting property. Specifically, fluorine is preferably used as the halogen group because it is stabile. Note that an organic compound having an electron-accepting property is easily deposited by evaporation and its film can be easily formed. As a result, the productivity of the light-emitting device can be increased.

Specifically, it is possible to use, for example, 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene) malononitrile.

A compound in which electron-withdrawing groups are bonded to a condensed aromatic ring having a plurality of heteroatoms, such as HAT-CN, is particularly preferable because it is thermally stable.

### [Example of Organic Compound AM1]

The organic compound AM1 has a lowest unoccupied molecular orbital (LUMO) level lower than or equal to −5.0 eV (see FIG. 1B). It is preferable that the organic compound AM1 contain fluorine.

Alternatively, a [3]radialene derivative having an electron-withdrawing group (in particular, a cyano group or a halogen group such as a fluoro group) is preferable because it has a very high electron-accepting property.

Specifically, it is possible to use, for example, α,α′,α″-1,2,3-α,α′,α″-1,2,3-cyclopropanetriylidenetris[4-cyano-2,3,5,6-tetrafluorobenzeneacetonitrile], cyclopropanetriylidenetris[2,6-dichloro-3,5-difluoro-4-(trifluoromethyl)benzeneacetonitrile], or α,α′,α″-1,2,3-cyclopropanetriylidenetris[2,3,4,5,6-pentafluorobenzeneacetonitrile].

### <<Structure Example 2 of Layer 104>>

The layer 104 has a hole mobility lower than or equal to 1×10⁻³ cm/Vs when the square root of the electric-field strength [V/cm] is 600. The layer 104 has a resistivity higher than or equal to 1×10⁴ [Ω·cm] and lower than or equal to 1×10⁷ [Ω·cm]. The resistivity of the layer 104 is preferably higher than or equal to 5×10⁴ [Ω·cm] and lower than or equal to 1×10⁷ [Ω·cm], further preferably higher than or equal to 1×10⁵ [Ω·cm] and lower than or equal to 1×10⁷ [Ω·cm].

Here, in consideration of the crosstalk suppression effect, the resistivity of the layer 104 in the light-emitting device of one embodiment of the present invention is preferably as high as possible. However, it was found that too high a resistivity prevents hole injection and does not lead to a light-emitting device with a favorable lifetime. Therefore, the resistivity of a material included in the layer 104 is preferably higher than or equal to 1×10⁴ [Ω·cm] and lower than or equal to 1×10⁷ [Ω·cm]. The light-emitting device has a favorable lifetime, and a light-emitting apparatus that includes the light-emitting device can have favorable display quality with suppressed crosstalk.

The resistivity is preferably higher than or equal to 5×10⁴ [[Ω·cm] and lower than or equal to 1×10⁷ (Ω·cm], further preferably higher than or equal to 1×10⁵ [Ω·cm] and lower than or equal to 1× 10⁷ [Ω·cm] in terms of the crosstalk suppression effect.

### [Example of Organic Compound HM1]

As the organic compound HM1, for example, a compound having an aromatic amine skeleton, a carbazole derivative, an aromatic hydrocarbon, an aromatic hydrocarbon having a vinyl group, a high molecular compound (such as an oligomer, a dendrimer, or a polymer), or the like can be used.

The organic compound HM1 can be a substance having a relatively deep HOMO level. The organic compound HM1 has a HOMO level HOMO2. The HOMO level HOMO2 is higher than or equal to −5.7 eV and lower than or equal to −5.2 eV, preferably higher than or equal to −5.7 eV and lower than or equal to −5.3 eV, further preferably higher than or equal to −5.7 eV and lower than or equal to −5.4 eV (see FIG. 1B). In that case, hole injection to the unit 103 can be facilitated. Alternatively, hole injection to the layer 112 can be facilitated. Induction of holes can be moderately inhibited. Furthermore, the resistivity of the layer 104 can be increased to be in a favorable range. A cross talk phenomenon between adjacent light-emitting devices can be inhibited.

As an organic compound having a relatively deep HOMO level, for example, N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBANβNB), 4,4′-diphenyl-4″-(7; l′-binaphthyl-2-yl)triphenylamine (abbreviation: BBAαNβNB-03), 4,4′-diphenyl-4″-(7-phenyl) naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl) biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl) biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis([1,1′-biphenyl]-4-yl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF (4)), N-(1,1′-biphenyl-2-yl)-N-(9,9-dimethyl-9H-fluoren-2-yl)-9,9′-spirobi[9H-fluoren]-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]spiro-9,9′-bifluoren-2-amine (abbreviation: PCBASF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), or the like can be used.

### <<Structure Example of Layer 112>>

The layer 112 is held between the layer 104 and the layer 111 and has a single-layer structure or a stacked-layer structure. The layer 112 is in contact with the layer 104 (see FIG. 1A).

The layer 112 contains an organic compound HM2. For example, a material having a hole-transport property can be used for the layer 112. The layer 112 can be referred to as a hole-transport layer. A material having a wider band gap than the light-emitting material contained in the layer 111 is preferably used for the layer 112. In that case, energy transfer from excitons generated in the layer 111 to the layer 112 can be inhibited.

### [Material Having Hole-Transport Property]

A material having a hole mobility higher than or equal to 1×10⁻⁶ cm²/Vs can be suitably used as the material having a hole-transport property.

For example, a material with a hole-transport property that can be used for the layer 111 can be used for the layer 112. Specifically, a material with a hole-transport property that can be used as the host material can be used for the layer 112.

### [Example of Organic Compound HM2]

The organic compound HM2 has a HOMO level HOMO3. The HOMO level HOMO3 differs from the HOMO level HOMO2 by higher than or equal to −0.2 eV and lower than or equal to 0 eV (see FIG. 1B).

Accordingly, transfer of holes from the electrode 551 toward the layer 111 can be facilitated. Furthermore, a region contributing to light emission near the layer 111 can be moderately expanded toward the layer 113. The distribution of excitons generated by carrier recombination can be expanded in the thickness direction. The organic compound in an excited state can be inhibited from changing in properties. The reliability of the layer 111 can be increased. Consequently, a novel light-emitting device that is highly convenient, useful, or reliable can be provided.

Note that this embodiment can be combined with any of the other embodiments described in this specification as appropriate.

### Embodiment 2

In this embodiment, a structure of the light-emitting device 550 of one embodiment of the present invention is described with reference to FIG. 1A.

### <Structure Example of Light-Emitting Device 550>

The light-emitting device 550 described in this embodiment includes the electrode 551, the electrode 552, the unit 103, and a layer 105. The electrode 552 includes a region overlapping with the electrode 551, and the unit 103 includes a region held between the electrode 551 and the electrode 552. The layer 105 includes a region held between the unit 103 and the electrode 552. For example, the structure described in Embodiment 1 can be used for the unit 103.

### <Structure Example of Electrode 552>

A conductive material can be used for the electrode 552, for example. Specifically, a single layer or a stacked layer of a metal, an alloy, or a material containing a conductive compound can be used for the electrode 552.

For example, the material that can be used for the electrode 551 described in Embodiment 1 can be used for the electrode 552. In particular, a material having a lower work function than the electrode 551 can be favorably used for the electrode 552. Specifically, a material having a work function lower than or equal to 3.8 eV is preferable.

For example, an element belonging to Group 1 of the periodic table, an element belonging to Group 2 of the periodic table, a rare earth metal, or an alloy containing any of these elements can be used for the electrode 552.

Specifically, lithium (Li), cesium (Cs), or the like; magnesium (Mg), calcium (Ca), strontium (Sr), or the like; europium (Eu), ytterbium (Yb), or the like; or an alloy containing any of these (MgAg or AlLi) can be used for the electrode 552.

### <<Structure Example of Layer 105>>

A material having an electron-injection property can be used for the layer 105, for example. The layer 105 can be referred to as an electron-injection layer.

Specifically, a substance having a donor property can be used for the layer 105. Alternatively, a material in which a substance having a donor property and a material having an electron-transport property are combined can be used for the layer 105. Alternatively, electride can be used for the layer 105. This can facilitate injection of electrons from the electrode 552, for example. Alternatively, besides a material having a low work function, a material having a high work function can also be used for the electrode 552. Alternatively, a material used for the electrode 552 can be selected from a wide range of materials regardless of its work function. Specifically, Al, Ag, ITO, indium oxide-tin oxide containing silicon or silicon oxide, or the like can be used for the electrode 552. Alternatively, the driving voltage of the light-emitting device can be lowered.

### [Substance Having Donor Property]

For example, an alkali metal, an alkaline earth metal, a rare earth metal, or a compound thereof (an oxide, a halide, a carbonate, or the like) can be used as the substance having a donor property. Alternatively, an organic compound such as tetrathianaphthacene (abbreviation: TTN), nickelocene, or decamethylnickelocene can be used as the substance having a donor property.

As an alkali metal compound (including an oxide, a halide, and a carbonate), lithium oxide, lithium fluoride (LiF), cesium fluoride (CsF), lithium carbonate, cesium carbonate, 8-hydroxyquinolinato-lithium (abbreviation: Liq), or the like can be used.

As an alkaline earth metal compound (including an oxide, a halide, and a carbonate), calcium fluoride (CaF₂) or the like can be used.

### [Structure Example 1 of Composite Material]

A material in which a plurality of kinds of substances are combined can be used as the material having an electron-injection property. For example, a substance having a donor property and a material having an electron-transport property can be used for the composite material.

[Material having electron-transport property]

For example, a metal complex or an organic compound having π-electron deficient heteroaromatic ring skeleton can be used as the material having an electron-transport property.

For example, a material having an electron-transport property usable for the unit 103 can be used for the composite material.

### [Structure Example 2 of Composite Material]

A material including a fluoride of an alkali metal in a microcrystalline state and a material having an electron-transport property can be used for the composite material. Alternatively, a material including a fluoride of an alkaline earth metal in a microcrystalline state and a material having an electron-transport property can be used for the composite material. In particular, a composite material containing a fluoride of an alkali metal or a fluoride of an alkaline earth metal at higher than or equal to 50 wt % can be suitably used. Alternatively, a composite material including an organic compound having a bipyridine skeleton can be suitably used. In that case, the refractive index of the layer 105 can be reduced. Alternatively, the external quantum efficiency of the light-emitting device can be improved.

### [Structure Example 3 of Composite Material]

For example, a composite material containing a first organic compound having an unshared electron pair and a first metal can be used for the layer 105. The sum of the number of electrons of the first organic compound and the number of electrons of the first metal is preferably an odd number. The molar ratio of the first metal to 1 mol of the first organic compound is preferably greater than or equal to 0.1 and less than or equal to 10, further preferably greater than or equal to 0.2 and less than or equal to 2, still further preferably greater than or equal to 0.2 and less than or equal to 0.8.

Accordingly, the first organic compound having an unshared electron pair interacts with the first metal and thus can form a singly occupied molecular orbital (SOMO). Furthermore, in the case where electrons are injected from the electrode 552 into the layer 105, a barrier therebetween can be lowered. The first metal has a low reactivity with water and oxygen; thus, the moisture resistance of the light-emitting device can be improved.

For the layer 105, a composite material that allows the spin density measured by an electron spin resonance method (ESR) to be preferably higher than or equal to 1×10¹⁶ spins/cm³, further preferably higher than or equal to 5×10¹⁶ spins/cm³, still further preferably higher than or equal to 1×10¹⁷ spins/cm³ can be used.

### [Organic Compound Having Unshared Electron Pair]

For example, a material having an electron-transport property can be used for the organic compound having an unshared electron pair. For example, a compound having an electron deficient heteroaromatic ring can be used. Specifically, a compound having at least one of a pyridine ring, a diazine ring (a pyrimidine ring, a pyrazine ring, and a pyridazine ring), and a triazine ring can be used. Accordingly, the driving voltage of the light-emitting device can be reduced.

Note that the lowest unoccupied molecular orbital (LUMO) of the organic compound having an unshared electron pair is preferably greater than or equal to −3.6 eV and less than or equal to −2.3 eV. In general, the HOMO level and the LUMO level of an organic compound can be estimated by CV (cyclic voltammetry), photoelectron spectroscopy, optical absorption spectroscopy, inverse photoelectron spectroscopy, or the like.

For example, 4,7-diphenyl-1,10-phenanthroline (abbreviation: BPhen), 2,9-bis(naphthalen-2-yl)-4,7-diphenyl-1,10-phenanthroline (abbreviation: NBPhen), diquinoxalino[2,3-a: 2′,3′-c]phenazine (abbreviation: HATNA), 2,4,6-tris[3′-(pyridin-3-yl) biphenyl-3-yl]-1,3,5-triazine (abbreviation: TmPPPyTz), or the like can be used as the organic compound having an unshared electron pair. Note that NBPhen has a higher glass transition temperature (Tg) than BPhen and thus has high heat resistance.

Alternatively, for example, copper phthalocyanine can be used for the organic compound having an unshared electron pair. The number of electrons of the copper phthalocyanine is an odd number.

### [First Metal]

For example, when the number of electrons of the first organic compound having an unshared electron pair is an even number, a composite material of a metal that belongs to an odd-numbered group in the periodic table and the first organic compound can be used for the layer 105.

For example, manganese (Mn), which is a metal belonging to Group 7, cobalt (Co), which is a metal belonging to Group 9, copper (Cu), silver (Ag), and gold (Au), which are metals belonging to Group 11, aluminum (Al) and indium (In), which are metals belonging to Group 13 are odd-numbered groups in the periodic table. Note that elements belonging to Group 11 have a lower melting point than elements belonging to Group 7 or Group 9 and thus are suitable for vacuum evaporation. In particular, Ag is preferable because of its low melting point.

The use of Ag for the electrode 552 and the layer 105 can increase the adhesion between the layer 105 and the electrode 552.

When the number of electrons of the first organic compound having an unshared electron pair is an odd number, a composite material of the first metal that belongs to an even-numbered group in the periodic table and the first organic compound can be used for the layer 105. For example, iron (Fe), which is a metal belonging to Group 8, is an element belonging to an even-numbered group in the periodic table.

### [Electride]

For example, a substance obtained by adding electrons at high concentration to an oxide where calcium and aluminum are mixed, or the like can be used as the material having an electron-injection property.

Note that this embodiment can be combined with any of the other embodiments described in this specification as appropriate.

### Embodiment 3

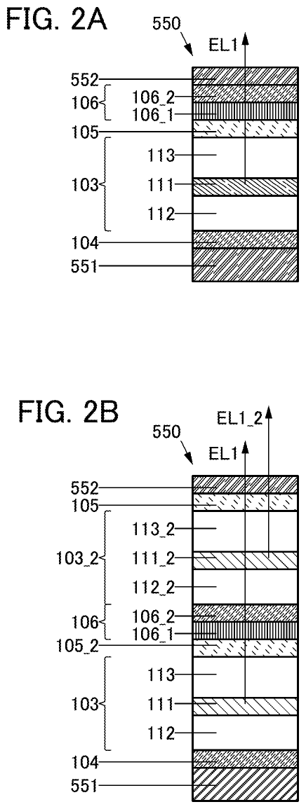

In this embodiment, a structure of the light-emitting device 550 of one embodiment of the present invention is described with reference to FIG. 2A.

FIG. 2A is a cross-sectional view illustrating a structure of the light-emitting device of one embodiment of the present invention.

### <Structure Example of Light-Emitting Device 550>

The light-emitting device 550 described in this embodiment includes the electrode 551, the electrode 552, the unit 103, and an intermediate layer 106 (see FIG. 2A). The electrode 552 includes a region overlapping with the electrode 551, and the unit 103 includes a region held between the electrode 551 and the electrode 552. The intermediate layer 106 includes a region held between the unit 103 and the electrode 552.

### <<Structure Example of Intermediate Layer 106>>

The intermediate layer 106 includes a layer 106_1 and a layer 106_2. The layer 106_2 includes a region held between the layer 106_1 and the electrode 552.

### <<Structure Example of Layer 106_1>>

For example, a material having an electron-transport property can be used for the layer 106_1. The layer 106_1 can be referred to as an electron-relay layer. With the use of the layer 106_1, a layer that is in contact with the anode side of the layer 106_1 can be distanced from a layer that is in contact with the cathode side of the layer 106_1. It is possible to reduce interaction between the layer in contact with the anode side of the layer 106_1 and the layer in contact with the cathode side of the layer 106_1. Electrons can be smoothly supplied to the layer in contact with the anode side of the layer 106_1.

A substance whose LUMO level is positioned between the LUMO level of the substance having an electron-accepting property included in the layer in contact with the anode side of the layer 106_1 and the LUMO level of the substance included in the layer in contact with the cathode side of the layer 106_1 can be suitably used for the layer 106_1.

For example, a material that has a LUMO level higher than or equal to −5.0 eV, preferably higher than or equal to −5.0 eV and lower than or equal to −3.0 eV can be used for the layer 106_1.

Specifically, a phthalocyanine-based material can be used for the layer 106_1. Alternatively, a metal complex having a metal-oxygen bond and an aromatic ligand can be used for the layer 106_1.

### <<Structure Example of Layer 106_2>>

For example, a material that supplies electrons to the anode side and supplies holes to the cathode side when voltage is applied can be used for the layer 106_2. Specifically, electrons can be supplied to the unit 103 that is positioned on the anode side. The layer 106_2 can be referred to as a charge-generation layer.

Specifically, a material with a hole-injection property that can be used for the layer 104 can be used for the layer 106_2. For example, a composite material can be used for the layer 106_2. For another example, a stacked film in which a film including the composite material and a film including a material having a hole-transport property are stacked can be used as the layer 106_2.

Note that this embodiment can be combined with any of the other embodiments described in this specification as appropriate.

### Embodiment 4

In this embodiment, a structure of the light-emitting device 550 of one embodiment of the present invention is described with reference to FIG. 2B.

FIG. 2B is a cross-sectional view illustrating a structure of the light-emitting device of one embodiment of the present invention, which is different from the structure illustrated in FIG. 2A.

### <Structure Example of Light-Emitting Device 550>

The light-emitting device 550 described in this embodiment includes the electrode 551, the electrode 552, the unit 103, the intermediate layer 106, and a unit 103_2 (see FIG. 2B). The electrode 552 includes a region overlapping with the electrode 551. The unit 103 includes a region held between the electrode 551 and the electrode 552, the intermediate layer 106 includes a region held between the unit 103 and the electrode 552, and the unit 103_2 includes a region held between the intermediate layer 106 and the electrode 552. The unit 103_2 has a function of emitting light EL1_2. The light-emitting device 550 includes a layer 105_2, and the layer 105_2 includes a region held between the unit 103 and the intermediate layer 106.

In other words, the light-emitting device 550 includes the stacked units between the electrode 551 and the electrode 552. The number of stacked units is not limited to two, and three or more units can be stacked. A structure including the stacked units held between the electrode 551 and the electrode 552 and the intermediate layer 106 held between the units is referred to as a stacked light-emitting device or a tandem light-emitting device in some cases. This structure can provide light emission at high luminance while the current density is kept low. Alternatively, the reliability can be improved. Alternatively, the driving voltage can be lowered as compared with other structures with the same luminance. Furthermore, power consumption can be reduced.

### <<Structure Example 1 of Unit 103_2>>

The unit 103_2 includes a layer 111_2, a layer 112_2, and a layer 113_2. The structure usable for the unit 103 can be used for the unit 103_2. For example, the unit 103_2 can have the same structure as the unit 103.

### <<Structure Example 2 of Unit 103_2>>

Alternatively, a structure different from that of the unit 103 can be used for the unit 103_2. For example, a structure that exhibits an emission color different from the emission color of the unit 103 can be used for the unit 103_2. Specifically, the unit 103 that emits red light and green light and the unit 103_2 that emits blue light can be employed. Accordingly, a light-emitting device that emits light of a desired color can be provided. For example, a light-emitting device that emits white light can be provided.

### <<Structure Example of Intermediate Layer 106>>

The intermediate layer 106 has a function of supplying electrons to one of the unit 103 and the unit 103_2 and supplying holes to the other. For example, the intermediate layer 106 described in Embodiment 3 can be used.

### <<Structure Example of Layer 105_2>>

A material having an electron-injection property can be used for the layer 105_2, for example. The layer 105_2 can be referred to as an electron-injection layer. For example, the material that can be used for the layer 105 described in Embodiment 2 can be used for the layer 105_2.

### <Fabrication Method of Light-Emitting Device 550>

For example, each layer of the electrode 551, the electrode 552, the unit 103, the intermediate layer 106, and the unit 103_2 can be formed by a dry process, a wet process, an evaporation method, a droplet discharge method, a coating method, a printing method, or the like. Different methods can be used to form the components.

Specifically, the light-emitting device 550 can be manufactured with a vacuum evaporation machine, an inkjet machine, a spin coater, a coating machine, a gravure printing machine, an offset printing machine, a screen printing machine, or the like.

For example, the electrode can be formed by a wet process or a sol-gel method using a paste of a metal material. An indium oxide-zinc oxide film can be formed by a sputtering method using a target obtained by adding, to indium oxide, zinc oxide at higher than or equal to 1 wt % and lower than or equal to 20 wt %. An indium oxide film containing tungsten oxide and zinc oxide (IWZO) can be formed by a sputtering method using a target containing, with respect to indium oxide, tungsten oxide at higher than or equal to 0.5 wt % and lower than or equal to 5 wt % and zinc oxide at higher than or equal to 0.1 wt % and lower than or equal to 1 wt %.

Note that this embodiment can be combined with any of the other embodiments described in this specification as appropriate.

### Embodiment 5

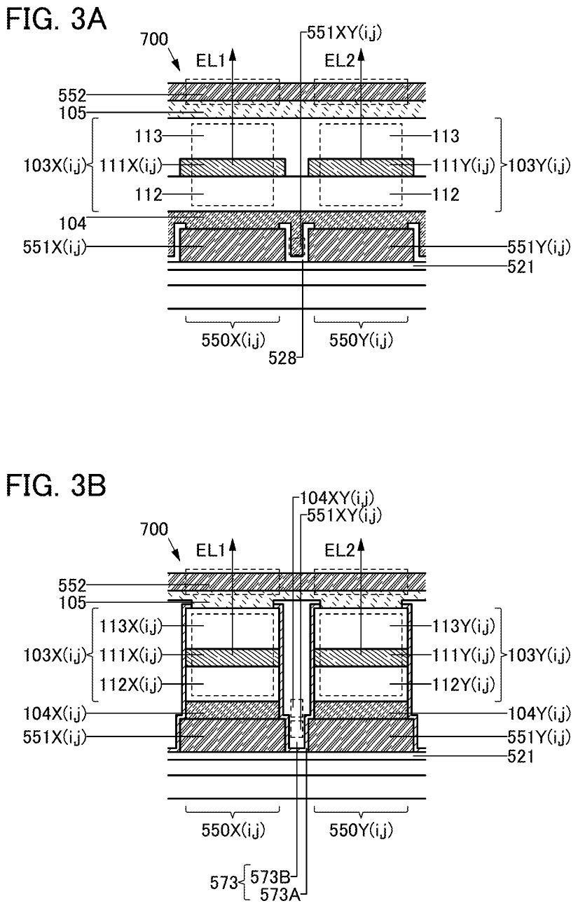

In this embodiment, a structure of a functional panel 700 of one embodiment of the present invention is described with reference to FIG. 3A and FIG. 3B.

FIG. 3A is a cross-sectional view illustrating a structure of the functional panel 700 of one embodiment of the present invention, and FIG. 3B is a cross-sectional view illustrating a structure of the functional panel 700 of one embodiment of the present invention different from that in FIG. 3A.

In this specification and the like, a device fabricated using a metal mask or an FMM (a fine metal mask or a high-resolution metal mask) may be referred to as a device having an MM (metal mask) structure. In this specification and the like, a device fabricated without using a metal mask or an FMM may be referred to as a device having an MML (metal maskless) structure.

### <Structure Example 1 of Functional Panel 700>

The functional panel 700 described in this embodiment includes a light-emitting device 550X(i,j) and a light-emitting device 550Y(i,j) (see FIG. 3A). The light-emitting device 550Y(i,j) is adjacent to the light-emitting device 550X(i,j).

The functional panel 700 further includes an insulating film 521, and the light-emitting device 550X(i,j) and the light-emitting device 550Y(i,j) are formed over the insulating film 521.

### <<Structure Example of Light-Emitting Device 550X(i,j)>>

The light-emitting device 550X(i,j) includes an electrode 551X(i,j), the electrode 552, and a unit 103X(i,j). Furthermore, the light-emitting device 550X(i,j) includes the layer 104 and the layer 105.

For example, the light-emitting device described in any of Embodiment 1 to Embodiment 4 can be used as the light-emitting device 550X(i,j). Specifically, the structure that can be used for the electrode 551 can be used for the electrode 551X(i,j). The structure that can be used for the unit 103 can be used for the unit 103X(i,j). The structure that can be used for the layer 104 and the structure that can be used for the layer 105 can be respectively used for the layer 104 and the layer 105.

### <<Structure Example 1 of Light-Emitting Device 550Y(i,j)>>

The light-emitting device 550Y(i,j) described in this embodiment includes an electrode 551Y(i,j), the electrode 552, and a unit 103Y(i,j) (see FIG. 3A). The electrode 552 includes a region overlapping with the electrode 551Y(i,j), and the unit 103Y(i,j) includes a region held between the electrode 551Y(i,j) and the electrode 552.

The electrode 551Y(i,j) is adjacent to the electrode 551X(i,j), and a gap 551XY(i,j) is provided between the electrode 551X(i,j) and the electrode 551Y(i,j).

For example, a material that can be used for the electrode 551X(i,j) can be used for the electrode 551Y(i,j). The potential supplied to the electrode 551Y(i,j) may be the same as or different from the potential supplied to the electrode 551X(i,j). By supplying a different potential, the light-emitting device 550Y(i,j) can be driven under conditions different from those for the light-emitting device 550X(i,j).

### <<Structure Example 1 of Unit 103Y(i,j)>>

The unit 103Y(i,j) has a single-layer structure or a stacked-layer structure.

For example, a layer selected from functional layers such as a light-emitting layer, a hole-transport layer, an electron-transport layer, and a carrier-blocking layer can be used in the unit 103Y(i,j). Moreover, a layer selected from functional layers such as a hole-injection layer, an electron-injection layer, an exciton-blocking layer, and a charge-generation layer can be used in the unit 103Y(i,j).

### <<Structure Example 2 of Unit 103Y(i,j)>>

For example, the unit 103Y(i,j) includes a layer 111Y(i,j), the layer 112, and the layer 113 (see FIG. 3A).

The layer 112 includes a region held between the electrode 551Y(i,j) and the layer 111Y(i,j), the layer 111Y(i,j) includes a region held between the layer 112 and the layer 113, and the layer 113 includes a region held between the layer 111Y(i,j) and the electrode 552.

### <<Structure Example 2 of Light-Emitting Device 550Y(i,j)>>

The light-emitting device 550Y(i,j) includes the layer 104 and the layer 105. The layer 104 includes a region held between the electrode 551Y(i,j) and the unit 103Y(i,j), and the layer 105 includes a region held between the unit 103Y(i,j) and the electrode 552.

Note that some of the components of the light-emitting device 550X(i,j) can be used as some of the components of the light-emitting device 550Y(i,j). Thus, some of the components can be used in common. Alternatively, the manufacturing process can be simplified.

### <<Structure Example 2 of Functional Panel 700>>

The functional panel 700 described in this embodiment includes an insulating film 528 (see FIG. 3A).

### <<Structure Example of Insulating Film 528>>

The insulating film 528 has openings; one opening overlaps with the electrode 551X(i,j) and the other opening overlaps with the electrode 551Y(i,j).

### <<Structure Example 3 of Functional Panel 700>>

The functional panel 700 described in this embodiment includes the light-emitting device 550X(i,j) and the light-emitting device 550Y(i,j), and the light-emitting device 550Y(i,j) is adjacent to the light-emitting device 550X(i,j) (see FIG. 3B).

The light-emitting device 550X(i,j) includes the electrode 551X(i,j), the electrode 552, and the unit 103X(i,j). Furthermore, the light-emitting device 550X(i,j) includes a layer 104X(i,j) and the layer 105. The structure that can be used for the layer 104 can be used for the layer 104X(i,j).

The light-emitting device 550Y(i,j) includes the electrode 551Y(i,j), the electrode 552, and the unit 103Y(i,j). The light-emitting device 550Y(i,j) further includes the layer 104Y(i,j) and the layer 105, and the gap 551XY(i,j) is provided between the electrode 551X(i,j) and the electrode 551Y(i,j).

The layer 104Y(i,j) is held between the electrode 551Y(i,j) and the electrode 552, the layer 104Y(i,j) is in contact with the electrode 551Y(i,j), and the layer 104Y(i,j) contains the organic compound HM1. A gap 104XY(i,j) is provided between the layer 104X(i,j) and the layer 104Y(i,j), and the gap 104XY(i,j) overlaps with the gap 551XY(i,j).

The light-emitting device 550Y(i,j) further includes the unit 103Y(i,j), and a gap is provided between the unit 103Y(i,j) and the light-emitting device 550X(i,j).

The functional panel is different from that described with reference to FIG. 3A in that the gap 104XY(i,j) is provided between the layer 104X(i,j) and the layer 104Y(i,j) and the unit 103Y(i,j) includes a gap between a layer 112X(i,j) and a layer 112Y(i,j) and a gap between a layer 113X(i,j) and a layer 113Y(i,j). Different portions are described in detail here, and the above description is referred to for portions that have similar structures.

### <<Structure Example of Layer 104Y(i,j)>>

A material having a hole-injection property can be used for the layer 104Y(i,j). The layer 104Y(i,j) can be referred to as a hole-injection layer. For example, the layer 104Y(i,j) contains the organic compound HM1 and the organic compound AM1. The gap 104XY(i,j) is provided between the layer 104Y(i,j) and the layer 104X(i,j). Accordingly, current flowing between the layer 104Y(i,j) and the layer 104X(i,j) can be drastically reduced.

### <<Structure Example 3 of Unit 103Y(i,j)>>

The unit 103Y(i,j) includes the layer 111Y(i,j), the layer 112Y(i,j), and the layer 113Y(i,j) (see FIG. 3B).

The layer 112Y(i,j) is held between the electrode 551Y(i,j) and the layer 111Y(i,j), and a gap is provided between the layer 112Y(i,j) and the layer 112X(i,j). Note that the structure that can be used for the layer 112 can be used for the layer 112Y(i,j).

The layer 111Y(i,j) is held between the layer 112Y(i,j) and the layer 113Y(i,j), and a gap is provided between the layer 111Y(i,j) and a layer 111X(i,j).

The layer 113Y(i,j) is held between the layer 111Y(i,j) and the electrode 552, and a gap is provided between the layer 113Y(i,j) and the layer 113X(i,j). Note that the structure that can be used for the layer 113 can be used for the layer 113Y(i,j).

In other words, a groove is provided between the unit 103X(i,j) and the unit 103Y(i,j), and the unit 103Y(i,j) has a sidewall along the groove. The unit 103X(i,j) also has a sidewall along the groove, and the sidewalls face each other.

### <Structure Example 4 of Functional Panel 700>

The functional panel 700 described in this embodiment includes an insulating film 573, for example (see FIG. 3B).

### <<Structure Example of Insulating Film 573>>

The insulating film 573 includes an insulating film 573A and an insulating film 573B.

The insulating film 573A includes a region held between the insulating film 573B and the insulating film 521, and the insulating film 573A is in contact with the insulating film 521. The insulating film 573A includes a region in contact with the sidewall of the unit 103Y(i,j) and a region in contact with the sidewall of the unit 103X(i,j).

### <Structure Example 5 of Functional Panel 700>

The functional panel 700 described in this embodiment includes the layer 111Y(i,j) (see FIG. 3A or FIG. 3B).

### <<Structure Example 1 of Layer 111Y(i,j)>>

A light-emitting material or a light-emitting material and a host material can be used for the layer 111Y(i,j), for example. The layer 111Y(i,j) can be referred to as a light-emitting layer. The layer 111Y(i,j) is preferably provided in a region where holes and electrons are recombined. In that case, energy generated by recombination of carriers can be efficiently converted into light and emitted. Furthermore, the layer 111Y(i,j) is preferably provided apart from a metal used for the electrode or the like. In that case, a quenching phenomenon caused by the metal used for the electrode or the like can be inhibited.

For example, a light-emitting material different from the light-emitting material used for the layer 111X(i,j) can be used for the layer 111Y(i,j). Specifically, a light-emitting material whose emission color is different from the emission color of the layer 111X(i,j) can be used for the layer 111Y(i,j). Thus, light-emitting devices with different hues can be provided. Alternatively, additive color mixing can be performed using a plurality of light-emitting devices with different hues. Alternatively, it is possible to express a color of a hue that an individual light-emitting device cannot display.

For example, a light-emitting device that emits blue light, a light-emitting device that emits green light, and a light-emitting device that emits red light can be provided in the functional panel 700. Alternatively, a light-emitting device that emits white light, a light-emitting device that emits yellow light, and a light-emitting device that emits infrared rays can be provided in the functional panel 700.

### <<Structure Example 2 of Layer 111Y(i,j)>>

For example, a fluorescent substance, a phosphorescent substance, or a substance exhibiting thermally activated delayed fluorescence TADF (also referred to as a TADF material) can be used as the light-emitting material. Thus, energy generated by recombination of carriers can be released as light EL2 from the light-emitting material (see FIG. 3A or FIG. 3B).

### [Fluorescent Substance]

For example, a fluorescent substance that can be used for the layer 111 can be used for the layer 111Y(i,j). Note that without being limited thereto, any of a variety of known fluorescent substances can be used for the layer 111Y(i,j).

### [Phosphorescent Substance]

For example, a phosphorescent substance that can be used for the layer 111 can be used for the layer 111Y(i,j). Note that without being limited to thereto, any of a variety of known phosphorescent substances can be used for the layer 111Y(i,j).

### [Substance Exhibiting Thermally Activated Delayed Fluorescence (TADF)]

For example, a TADF material that can be used for the layer 111 can be used for the layer 111Y(i,j). Note that without being limited thereto, any of a variety of known TADF materials can be used for the layer 111Y(i,j).

### <<Structure Example 3 of Layer 111Y(i,j)>>

A material having a carrier-transport property can be used as the host material. For example, a material having a hole-transport property, a material having an electron-transport property, a substance exhibiting thermally activated delayed fluorescence TADF, a material having an anthracene skeleton, or a mixed material can be used as the host material. A material having a wider band gap than the light-emitting material contained in the layer 111Y(i,j) is preferably used as the host material. In that case, energy transfer from excitons generated in the layer 111Y(i,j) to the host material can be inhibited.

For example, a host material that can be used for the layer 111 can be used for the layer 111Y(i,j).

### <<Structure Example of Layer 112Y(i,j)>>

A material having a hole-transport property can be used for the layer 112Y(i,j), for example. The layer 112Y(i,j) can be referred to as a hole-transport layer. A material having a wider band gap than the light-emitting material contained in the layer 111Y(i,j) is preferably used for the layer 112Y(i,j). In that case, energy transfer from excitons generated in the layer 111Y(i,j) to the layer 112Y(i,j) can be inhibited.

### [Material Having Hole-Transport Property]

A material having a hole mobility higher than or equal to 1×10⁻⁶ cm²/Vs can be suitably used as the material having a hole-transport property.

For example, a material with a hole-transport property that can be used for the layer 111 can be used for the layer 112Y(i,j). Specifically, a material with a hole-transport property that can be used as the host material can be used for the layer 112Y(i,j).

### <<Structure Example of Layer 113Y(i,j)>>

A material having an electron-transport property, a material having an anthracene skeleton, a mixed material, or the like can be used for the layer 113Y(i,j), for example. The layer 113Y(i,j) can be referred to as an electron-transport layer. A material having a wider band gap than the light-emitting material contained in the layer 111Y(i,j) is preferably used for the layer 113Y(i,j). In that case, energy transfer from excitons generated in the layer 111Y(i,j) to the layer 113Y(i,j) can be inhibited.

### [Material Having Electron-Transport Property]

For example, a metal complex or an organic compound having a π-electron deficient heteroaromatic ring skeleton can be used as the material having an electron-transport property.

For example, a material with an electron-transport property that can be used for the layer 111Y(i,j) can be used for the layer 113Y(i,j). Specifically, a material with an electron-transport property that can be used as the host material can be used for the layer 113Y(i,j).

Note that this embodiment can be combined with any of the other embodiments described in this specification as appropriate.

### Embodiment 6

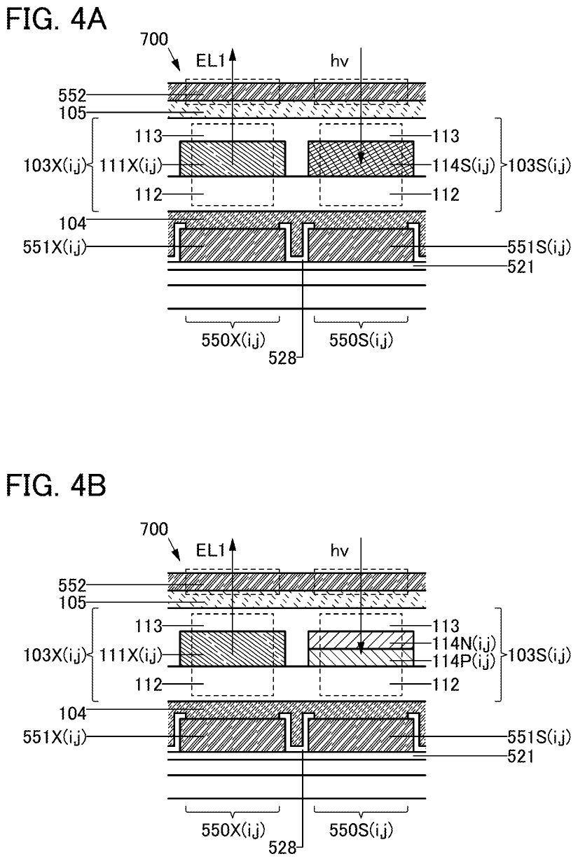

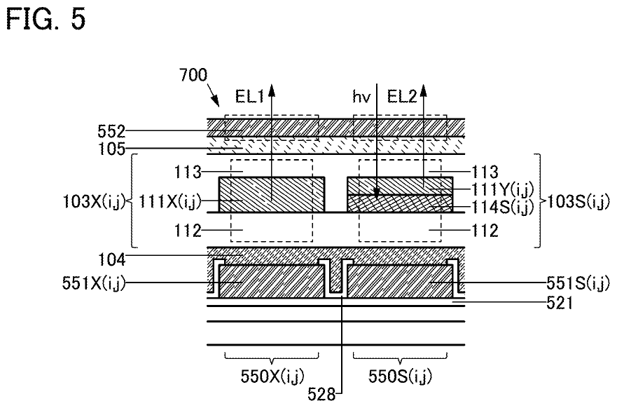

In this embodiment, a structure of the functional panel 700 of one embodiment of the present invention is described with reference to FIG. 4 and FIG. 5.

FIG. 4A is a cross-sectional view illustrating a structure of the functional panel 700 of one embodiment of the present invention, and FIG. 4B is a cross-sectional view illustrating a structure of the functional panel 700 of one embodiment of the present invention different from that in FIG. 4A.

FIG. 5 is a cross-sectional view illustrating a structure of the functional panel 700 of one embodiment of the present invention.

### <Structure Example 1 of Functional Panel 700>

The functional panel 700 described in this embodiment includes the light-emitting device 550X(i,j) and an optical functional device 550S(i,j) (see FIG. 4A).

For example, the light-emitting device described in any of Embodiment 1 to Embodiment 4 can be used as the light-emitting device 550X(i,j).

### <Structure Example of Optical Functional Device 550S(i,j)>

The optical functional device 550S(i,j) described in this embodiment includes an electrode 551S(i,j), the electrode 552, and a unit 103S(i,j). The electrode 552 includes a region overlapping with the electrode 551S(i,j), and the unit 103S(i,j) includes a region held between the electrode 551S(i,j) and the electrode 552.

The optical functional device 550S(i,j) includes the layer 104 and the layer 105. The layer 104 includes a region held between the electrode 551S(i,j) and the unit 103S(i,j), and the layer 105 includes a region held between the unit 103S(i,j) and the electrode 552. Note that some of the components of the light-emitting device 550X(i,j) can be used as some of the components of the optical functional device 550S(i,j). Thus, some of the components can be used in common. Alternatively, the manufacturing process can be simplified.

### <Structure Example 1 of Unit 103S(i,j)>

The unit 103S(i,j) has a single-layer structure or a stacked-layer structure. For example, the unit 103S(i,j) includes a layer 114S(i,j), the layer 112, and the layer 113 (see FIG. 4A).

The layer 114S(i,j) includes a region held between the layer 112 and the layer 113, the layer 112 includes a region held between the electrode 551S(i,j) and the layer 114S(i,j), and the layer 113 includes a region held between the layer 114S(i,j) and the electrode 552.

For example, a layer selected from functional layers such as a photoelectric conversion layer, a hole-transport layer, an electron-transport layer, and a carrier-blocking layer can be used in the unit 103S(i,j). Moreover, a layer selected from functional layers such as an exciton-blocking layer and a charge-generation layer can be used in the unit 103S(i,j).

The unit 103S(i,j) absorbs light hv, supplies electrons to one electrode, and supplies holes to the other electrode. For example, the unit 103S(i,j) supplies holes to the electrode 551S(i,j) and supplies electrons to the electrode 552.

### <<Structure Example of Layer 112>>

A material having a hole-transport property can be used for the layer 112, for example. The layer 112 can be referred to as a hole-transport layer. For example, the structure described in Embodiment 1 can be used for the layer 112.

### <<Structure Example of Layer 113>>

A material having an electron-transport property, a material having an anthracene skeleton, or a mixed material can be used for the layer 113, for example. For example, the structure described in Embodiment 1 can be used for the layer 113.

### <<Structure Example 1 of Layer 114S(i,j)>>

For example, an electron-accepting material and an electron-donating material can be used for the layer 114S(i,j). Specifically, a material that can be used for an organic solar cell can be used for the layer 114S(i,j). The layer 114S(i,j) can be referred to as a photoelectric conversion layer. The layer 114S(i,j) absorbs the light hv, supplies electrons to one electrode, and supplies holes to the other electrode. For example, the layer 114S(i,j) supplies holes to the electrode 551S(i,j) and supplies electrons to the electrode 552.

### [Example of Electron-Accepting Material]

As the electron-accepting material, a fullerene derivative or a non-fullerene electron acceptor can be used, for example.

As the electron-accepting material, C₆₀ fullerene, C₇₀ fullerene, [6,6]-Phenyl-C71-butyric acid methyl ester (abbreviation: PC70BM), [6,6]-Phenyl-C61-butyric acid methyl ester (abbreviation: PC60BM), 1′,1″,4′,4″-Tetrahydro-di[1,4]methanonaphthaleno[1,2:2′,3′,56,60:2″,3″][5,6]fullerene-C60 (abbreviation: ICBA), or the like can be used.

As the non-fullerene electron acceptor, a perylene derivative, a compound having a dicyanomethyleneindanone group, or the like can be used. For example, N,N′-dimethyl-3,4,9,10-perylenedicarboximide (abbreviation: Me-PTCDI) can be used.

### [Example of Electron-Donating Material]

As the electron-donating material, for example, a phthalocyanine compound, a tetracene derivative, a quinacridone derivative, or a rubrene derivative can be used.

As the electron-donating material, copper (II) phthalocyanine (abbreviation: CuPc), tin (II) phthalocyanine (abbreviation: SnPc), zinc phthalocyanine (abbreviation: ZnPc), tetraphenyldibenzoperiflanthene (abbreviation: DBP), rubrene, or the like can be used.

### <<Structure Example 2 of Layer 114S(i,j)>>

The layer 114S(i,j) can have a single-layer structure or a stacked-layer structure, for example. Specifically, the layer 114S(i,j) can have a bulk heterojunction structure. Alternatively, the layer 114S(i,j) can have a heterojunction structure.

### [Structure Example of Mixed Material]

A mixed material containing an electron-accepting material and an electron-donating material can be used for the layer 114S(i,j), for example. Note that a structure in which such a mixed material containing an electron-accepting material and an electron-donating material is used for the layer 114S(i,j) can be referred to as a bulk heterojunction structure.

Specifically, a mixed material containing C₇₀ fullerene and DBP can be used for the layer 114S(i,j).

### [Example of Heterojunction Structure]

A layer 114N(i,j) and a layer 114P(i,j) can be used for the layer 114S(i,j). The layer 114N(i,j) includes a region held between one electrode and the layer 114P(i,j), and the layer 114P(i,j) includes a region held between the layer 114N(i,j) and the other electrode. For example, the layer 114N(i,j) includes a region held between the electrode 552 and the layer 114P(i,j), and the layer 114P(i,j) includes a region held between the layer 114N(i,j) and the electrode 551S(i,j) (see FIG. 4B).

An n-type semiconductor can be used for the layer 114N(i,j). For example, Me-PTCDI can be used for the layer 114N(i,j).

A p-type semiconductor can be used for the layer 114P(i,j). For example, rubrene can be used for the layer 114P(i,j).

Note that the optical functional device 550S(i,j) in which the layer 114P(i,j) is in contact with the layer 114N(i,j) can be referred to as a pn-junction photodiode.

### <Structure Example 2 of Unit 103S(i,j)>

The unit 103S(i,j) includes the layer 111Y(i,j), and the layer 111Y(i,j) includes a region held between the layer 114S(i,j) and the layer 113 (see FIG. 5).

Structure example 2 of the unit 103S(i,j) is different from Structure example 1 of the unit 103S(i,j) in that the layer 111Y(i,j) is provided. Different portions will be described in detail below, and the above description is referred to for portions having the same structure as the above.

### <<Structure Example of Layer 111Y(i,j)>>

A light-emitting material or a light-emitting material and a host material can be used for the layer 111Y(i,j), for example. The layer 111Y(i,j) can be referred to as a light-emitting layer. The layer 111Y(i,j) is preferably provided in a region where holes and electrons are recombined. In that case, energy generated by recombination of carriers can be efficiently converted into light and emitted. Furthermore, the layer 111Y(i,j) is preferably provided apart from a metal used for the electrode or the like. In that case, a quenching phenomenon caused by the metal used for the electrode or the like can be inhibited.

Specifically, the structure described in Embodiment 5 can be used for the layer 111Y(i,j). In particular, the structure that emits light with a wavelength which is hardly absorbed by the layer 114S(i,j) can be suitably used for the layer 111Y(i,j). Accordingly, the light EL2 emitted from the layer 111Y(i,j) can be extracted with high efficiency.

Note that this embodiment can be combined with any of the other embodiments described in this specification as appropriate.

### Embodiment 7

In this embodiment, a light-emitting apparatus that includes the light-emitting device described in any one of Embodiment 1 to Embodiment 4 is described.

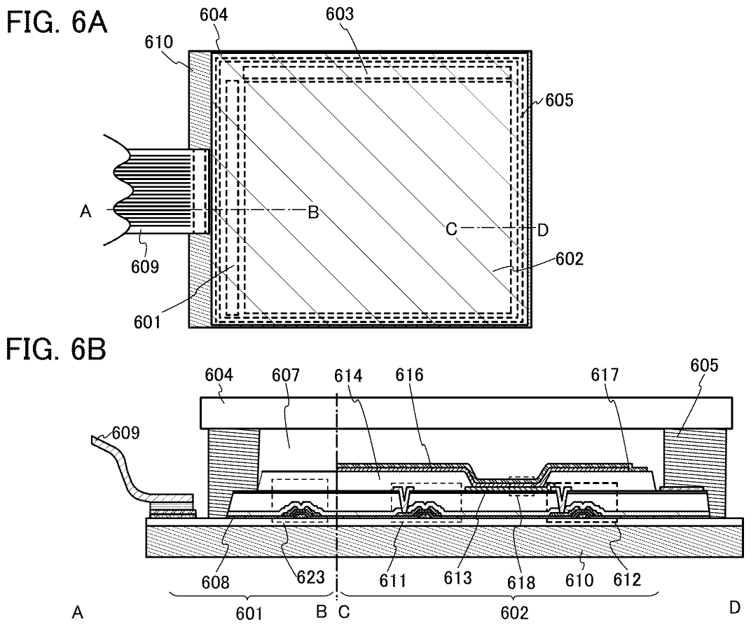

In this embodiment, a light-emitting apparatus fabricated using the light-emitting device described in any one of Embodiment 1 to Embodiment 4 is described with reference to FIG. 6. FIG. 6A is a top view illustrating the light-emitting apparatus, and FIG. 6B is a cross-sectional view taken along A-B and C-D in FIG. 6A. This light-emitting apparatus includes a driver circuit portion illustrated with dotted lines and a pixel portion 602, which are to control light emission of the light-emitting devices, and the driver circuit portion includes a source line driver circuit 601 and a gate line driver circuit 603. The light-emitting apparatus is provided with a sealing substrate 604 and a sealant 605, and a space 607 is surrounded by the sealant 605.

A lead wiring 608 is a wiring for transmitting signals to be input to the source line driver circuit 601 and the gate line driver circuit 603 and receives a video signal, a clock signal, a start signal, a reset signal, or the like from an FPC (flexible printed circuit) 609 serving as an external input terminal. Although only the FPC is illustrated here, a printed wiring board (PWB) may be attached to the FPC. The light-emitting apparatus in this specification includes, in its category, not only the light-emitting apparatus itself but also the light-emitting apparatus provided with the FPC or the PWB.

Next, a cross-sectional structure is described with reference to FIG. 6B. The driver circuit portions and the pixel portion are formed over an element substrate 610; here, the source line driver circuit 601, which is a driver circuit portion, and one pixel in the pixel portion 602 are illustrated.

The element substrate 610 may be formed using a substrate containing glass, quartz, an organic resin, a metal, an alloy, a semiconductor, or the like or a plastic substrate formed of FRP (Fiber Reinforced Plastics), PVF (polyvinyl fluoride), polyester, an acrylic resin, or the like.

There is no particular limitation on the structure of transistors used in pixels or driver circuits. For example, inverted staggered transistors may be used, or staggered transistors may be used. Furthermore, top-gate transistors or bottom-gate transistors may be used. A semiconductor material used for the transistors is not particularly limited, and for example, silicon, germanium, silicon carbide, gallium nitride, or the like can be used. Alternatively, an oxide semiconductor containing at least one of indium, gallium, and zinc, such as an In—Ga—Zn-based metal oxide, may be used.

There is no particular limitation on the crystallinity of a semiconductor material used for the transistors, and an amorphous semiconductor or a semiconductor having crystallinity (a microcrystalline semiconductor, a polycrystalline semiconductor, a single crystal semiconductor, or a semiconductor partly including crystal regions) may be used. A semiconductor having crystallinity is preferably used, in which case degradation of the transistor characteristics can be inhibited.

Here, an oxide semiconductor is preferably used for semiconductor devices such as the transistors provided in the pixels or the driver circuits and transistors used for after-mentioned touch sensors and the like. In particular, an oxide semiconductor having a wider band gap than silicon is preferably used. When an oxide semiconductor having a wider band gap than silicon is used, the off-state current of the transistors can be reduced.

The oxide semiconductor preferably contains at least indium (In) or zinc (Zn). Further preferably, the oxide semiconductor contains an oxide represented by an In-M-Zn-based oxide (M represents a metal such as Al, Ti, Ga, Ge, Y, Zr, Sn, La, Ce, or Hf).

As a semiconductor layer, it is particularly preferable to use an oxide semiconductor film including a plurality of crystal parts whose c-axes are aligned perpendicular to a surface on which the semiconductor layer is formed or the top surface of the semiconductor layer and having no grain boundary between adjacent crystal parts.

The use of such materials for the semiconductor layer makes it possible to provide a highly reliable transistor in which a change in the electrical characteristics is inhibited.

Charge accumulated in a capacitor through a transistor including the above-described semiconductor layer can be held for a long time because of the low off-state current of the transistor. When such a transistor is used in a pixel, operation of a driver circuit can be stopped while a gray scale of an image displayed in each display region is maintained. As a result, an electronic device with extremely low power consumption can be obtained.

For stable characteristics or the like of the transistor, a base film is preferably provided. The base film can be formed to be a single layer or a stacked layer using an inorganic insulating film such as a silicon oxide film, a silicon nitride film, a silicon oxynitride film, or a silicon nitride oxide film. The base film can be formed by a sputtering method, a CVD (Chemical Vapor Deposition) method (e.g., a plasma CVD method, a thermal CVD method, or an MOCVD (Metal Organic CVD) method), an ALD (Atomic Layer Deposition) method, a coating method, a printing method, or the like. Note that the base film does not have to be provided if not necessary.

Note that an FET 623 is illustrated as a transistor formed in the source line driver circuit 601. The driver circuit is formed with any of a variety of circuits such as a CMOS circuit, a PMOS circuit, or an NMOS circuit. Although a driver-integrated type in which the driver circuit is formed over the substrate is described in this embodiment, the driver circuit is not necessarily formed over the substrate, and the driver circuit can be formed outside, not over the substrate. The pixel portion 602 is formed with a plurality of pixels including a switching FET 611, a current control FET 612, and a first electrode 613 electrically connected to a drain of the current control FET 612; however, without being limited thereto, a pixel portion in which three or more FETs and a capacitor are combined may be employed.

Note that an insulator 614 is formed to cover an end portion of the first electrode 613. Here, the insulator 614 can be formed using a positive photosensitive acrylic resin film.

In order to improve the coverage with an EL layer or the like which is formed later, the insulator 614 is formed to have a curved surface with curvature at its upper or lower end portion. For example, in the case where a positive photosensitive acrylic resin is used as a material for the insulator 614, only the upper end portion of the insulator 614 preferably has a curved surface with a curvature radius (greater than or equal to 0.2 μm and less than or equal to 3 μm). As the insulator 614, either a negative photosensitive resin or a positive photosensitive resin can be used.

An EL layer 616 and a second electrode 617 are formed over the first electrode 613. Here, as a material used for the first electrode 613 functioning as an anode, a material with a high work function is desirably used. For example, a single-layer film of an ITO film, an indium tin oxide film containing silicon, an indium oxide film containing zinc oxide at higher than or equal to 2 wt % and lower than or equal to 20 wt %, a titanium nitride film, a chromium film, a tungsten film, a Zn film, a Pt film, or the like, a stacked layer of a titanium nitride film and a film containing aluminum as its main component, a three-layer structure of a titanium nitride film, a film containing aluminum as its main component, and a titanium nitride film, or the like can be used. The stacked-layer structure enables low wiring resistance, favorable ohmic contact, and a function as an anode.

The EL layer 616 is formed by any of a variety of methods such as an evaporation method using an evaporation mask, an inkjet method, and a spin coating method. The EL layer 616 has the structure described in any one of Embodiment 1 to Embodiment 4. As another material included in the EL layer 616, a low molecular compound or a high molecular compound (including an oligomer or a dendrimer) may be used.

As a material used for the second electrode 617, which is formed over the EL layer 616 and functions as a cathode, a material with a low work function (e.g., Al, Mg, Li, Ca, or an alloy or a compound thereof (MgAg, MgIn, AlLi, or the like)) is preferably used. Note that in the case where light generated in the EL layer 616 passes through the second electrode 617, it is preferable to use, for the second electrode 617, a stacked layer of a thin metal film and a transparent conductive film (e.g., ITO, indium oxide containing zinc oxide at higher than or equal to 2 wt % and lower than or equal to 20 wt %, indium tin oxide containing silicon, or zinc oxide (ZnO)).

Note that a light-emitting device is formed with the first electrode 613, the EL layer 616, and the second electrode 617. The light-emitting device is the light-emitting device described in any one of Embodiment 1 to Embodiment 4. A plurality of light-emitting devices are formed in the pixel portion, and the light-emitting apparatus of this embodiment may include both the light-emitting device described in any one of Embodiment 1 to Embodiment 4 and a light-emitting device having a different structure.

The sealing substrate 604 is attached to the element substrate 610 with the sealant 605, so that the light-emitting device 618 is provided in the space 607 surrounded by the element substrate 610, the sealing substrate 604, and the sealant 605. The space 607 is filled with a filler; it is filled with an inert gas (e.g., nitrogen or argon) in some cases, and filled with the sealant in other cases. It is preferable that the sealing substrate have a recessed portion provided with a desiccant, in which case degradation due to the influence of moisture can be inhibited.

Note that an epoxy-based resin or glass frit is preferably used for the sealant 605. Furthermore, these materials are preferably materials that transmit moisture and oxygen as little as possible. As the material used for the sealing substrate 604, in addition to a glass substrate and a quartz substrate, a plastic substrate formed of FRP (Fiber Reinforced Plastics), PVF (polyvinyl fluoride), polyester, an acrylic resin, or the like can be used.

Although not illustrated in FIG. 6A or FIG. 6B, a protective film may be provided over the second electrode. The protective film is formed using an organic resin film or an inorganic insulating film. The protective film may be formed so as to cover an exposed portion of the sealant 605. The protective film can be provided to cover surfaces and side surfaces of the pair of substrates and exposed side surfaces of a sealing layer, an insulating layer, and the like.

The protective film can be formed using a material that does not easily transmit an impurity such as water. Thus, diffusion of an impurity such as water from the outside into the inside can be effectively inhibited.

As a material of the protective film, an oxide, a nitride, a fluoride, a sulfide, a ternary compound, a metal, a polymer, or the like can be used. For example, it is possible to use a material containing aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, indium oxide, or the like; a material containing aluminum nitride, hafnium nitride, silicon nitride, tantalum nitride, titanium nitride, niobium nitride, molybdenum nitride, zirconium nitride, gallium nitride, or the like; or a material containing a nitride containing titanium and aluminum, an oxide containing titanium and aluminum, an oxide containing aluminum and zinc, a sulfide containing manganese and zinc, a sulfide containing cerium and strontium, an oxide containing erbium and aluminum, an oxide containing yttrium and zirconium, or the like.

The protective film is preferably formed using a film formation method with favorable step coverage. One of such methods is an atomic layer deposition (ALD) method. A material that can be formed by an ALD method is preferably used for the protective film. With the use of an ALD method, a dense protective film with reduced defects such as cracks or pinholes or with a uniform thickness can be formed. Furthermore, damage caused to a process member in forming the protective film can be reduced.

By an ALD method, for example, a uniform protective film with few defects can be formed even on a surface with a complex uneven shape or upper, side, and lower surfaces of a touch panel.

As described above, the light-emitting apparatus fabricated using the light-emitting device described in any one of Embodiment 1 to Embodiment 4 can be obtained.

The light-emitting apparatus in this embodiment is fabricated using the light-emitting device described in any one of Embodiment 1 to Embodiment 4 and thus can have favorable characteristics. Specifically, since the light-emitting device described in any one of Embodiment 1 to Embodiment 4 has favorable emission efficiency, the light-emitting apparatus can have low power consumption.

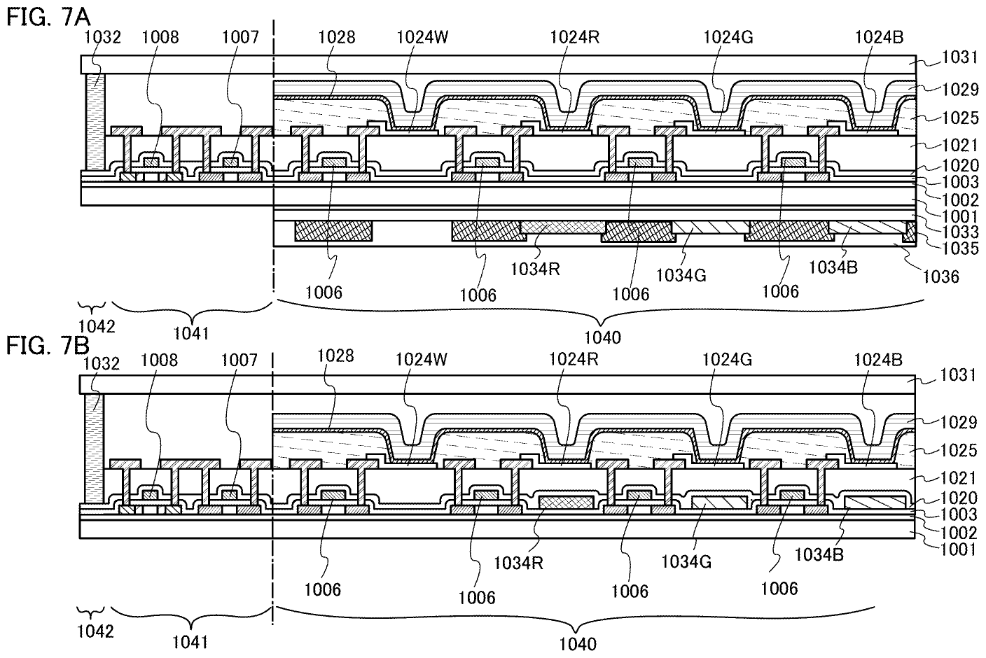

FIG. 7 illustrates examples of a light-emitting apparatus in which full color display is achieved by formation of light-emitting devices emitting white light and provision of coloring layers (color filters) and the like. FIG. 7A illustrates a substrate 1001; a base insulating film 1002; a gate insulating film 1003; a gate electrode 1006; a gate electrode 1007; a gate electrode 1008; a first interlayer insulating film 1020; a second interlayer insulating film 1021; a peripheral portion 1042; a pixel portion 1040; a driver circuit portion 1041; an electrode 1024W, an electrode 1024R, an electrode 1024G, and an electrode 1024B of the light-emitting devices; a partition 1025; an EL layer 1028; an electrode 1029 of the light-emitting devices; a sealing substrate 1031; a sealant 1032; and the like.

In FIG. 7A, coloring layers (a red coloring layer 1034R, a green coloring layer 1034G, and a blue coloring layer 1034B) are provided on a transparent base material 1033. A black matrix 1035 may be additionally provided. The transparent base material 1033 provided with the coloring layers and the black matrix is aligned and fixed to the substrate 1001. Note that the coloring layers and the black matrix 1035 are covered with an overcoat layer 1036. In FIG. 7A, a light-emitting layer from which light is emitted to the outside without passing through the coloring layer and light-emitting layers from which light is emitted to the outside, passing through the coloring layers of the respective colors are shown. Since light that does not pass through the coloring layer is white and light that passes through the coloring layer is red, green, or blue, an image can be expressed by pixels of the four colors.

FIG. 7B shows an example in which the coloring layers (the red coloring layer 1034R, the green coloring layer 1034G, and the blue coloring layer 1034B) are formed between the gate insulating film 1003 and the first interlayer insulating film 1020. As in the structure, the coloring layers may be provided between the substrate 1001 and the sealing substrate 1031.

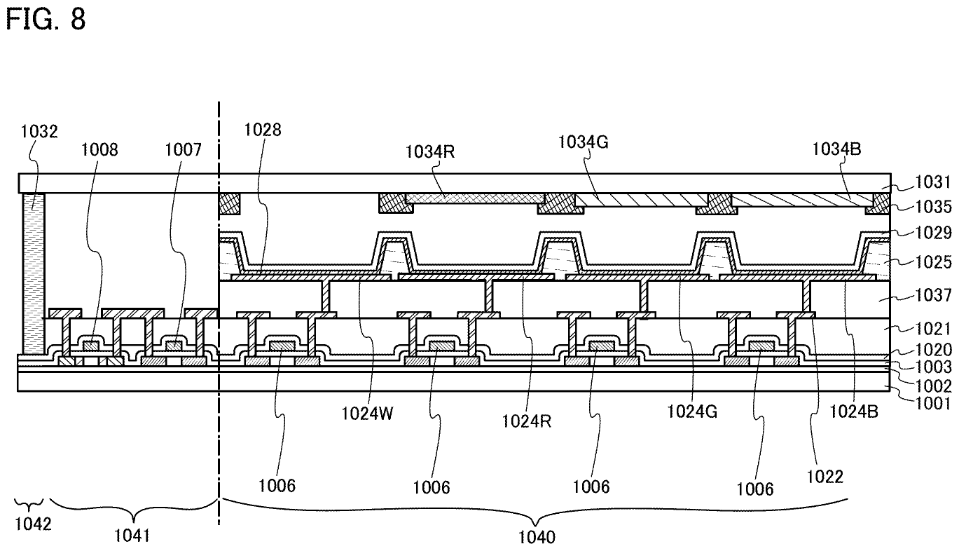

The above-described light-emitting apparatus has a structure in which light is extracted from the substrate 1001 side where FETs are formed (a bottom emission structure), but may have a structure in which light is extracted from the sealing substrate 1031 side (a top emission structure). FIG. 8 shows a cross-sectional view of a top-emission light-emitting apparatus. In this case, a substrate that does not transmit light can be used as the substrate 1001. The process up to the step of forming a connection electrode that connects the FET and the anode of the light-emitting device is performed in a manner similar to that of the light-emitting apparatus having a bottom emission structure. Then, a third interlayer insulating film 1037 is formed to cover an electrode 1022. This insulating film may have a planarization function. The third interlayer insulating film 1037 can be formed using a material similar to that of the second interlayer insulating film, and can alternatively be formed using any of other known materials.

The electrode 1024W, the electrode 1024R, the electrode 1024G, and the electrode 1024B of the light-emitting devices are each an anode here, but may each be a cathode. In the case of the top-emission light-emitting apparatus such as one in FIG. 8, the electrode 1024W, the electrode 1024R, the electrode 1024G, and the electrode 1024B are preferably reflective electrodes. The structure of the EL layer 1028 is such a structure as that of the unit 103 described in any one of Embodiment 1 to Embodiment 4, and an element structure with which white light emission can be obtained.

In the case of such a top-emission structure as in FIG. 8, sealing can be performed with the sealing substrate 1031 on which the coloring layers (the red coloring layer 1034R, the green coloring layer 1034G, and the blue coloring layer 1034B) are provided. The sealing substrate 1031 may be provided with the black matrix 1035 that is positioned between pixels. The coloring layers (the red coloring layer 1034R, the green coloring layer 1034G, and the blue coloring layer 1034B) or the black matrix may be covered with the overcoat layer 1036. Note that a substrate having a light-transmitting property is used as the sealing substrate 1031. Although an example in which full color display is performed using four colors of red, green, blue, and white is described here, there is no particular limitation and full color display may be performed using four colors of red, yellow, green, and blue or three colors of red, green, and blue.

In the top-emission light-emitting apparatus, a microcavity structure can be favorably employed. A light-emitting device with a microcavity structure can be obtained with the use of a reflective electrode as the first electrode and a transflective electrode as the second electrode. The light-emitting device with a microcavity structure includes at least an EL layer between the reflective electrode and the transflective electrode, and the EL layer includes at least a light-emitting layer serving as a light-emitting region.

Note that the reflective electrode is a film having a visible light reflectivity of 40% to 100%, preferably 70% to 100%, and a resistivity lower than or equal to 1×10⁻² Qcm. In addition, the transflective electrode is a film having a visible light reflectivity of 20% to 80%, preferably 40% to 70%, and a resistivity lower than or equal to 1×10⁻² Qcm.

Light emitted from the light-emitting layer included in the EL layer is reflected and resonated by the reflective electrode and the transflective electrode.

In the light-emitting device, by changing the thickness of the transparent conductive film, the above-described composite material, the carrier-transport material, or the like, the optical path length between the reflective electrode and the transflective electrode can be changed. Thus, light with a wavelength that is resonated between the reflective electrode and the transflective electrode can be intensified while light with a wavelength that is not resonated therebetween can be attenuated.

Note that light that is reflected back by the reflective electrode (first reflected light) considerably interferes with light that directly enters the transflective electrode from the light-emitting layer (first incident light). For this reason, the optical path length between the reflective electrode and the light-emitting layer is preferably adjusted to (2n−1)λ/4 (n is a natural number of 1 or larger and λ is a wavelength of light to be amplified). By adjusting the optical path length, the phases of the first reflected light and the first incident light can be aligned with each other and the light emitted from the light-emitting layer can be further amplified.

Note that in the above structure, the EL layer may include a plurality of light-emitting layers or may include a single light-emitting layer; for example, in combination with the structure of the above-described tandem light-emitting device, a plurality of EL layers each including a single or a plurality of light-emitting layer(s) may be provided in one light-emitting device with a charge-generation layer positioned between the EL layers.

With the microcavity structure, emission intensity with a specific wavelength in the front direction can be increased, whereby power consumption can be reduced. Note that in the case of a light-emitting apparatus that displays images with subpixels of four colors, red, yellow, green, and blue, the luminance can be increased owing to yellow light emission and each subpixel can employ a microcavity structure suitable for wavelengths of the corresponding color, so that the light-emitting apparatus can have favorable characteristics.

The light-emitting apparatus in this embodiment is fabricated using the light-emitting device described in any one of Embodiment 1 to Embodiment 4 and thus can have favorable characteristics. Specifically, since the light-emitting device described in any one of Embodiment 1 to Embodiment 4 has favorable emission efficiency, the light-emitting apparatus can have low power consumption.

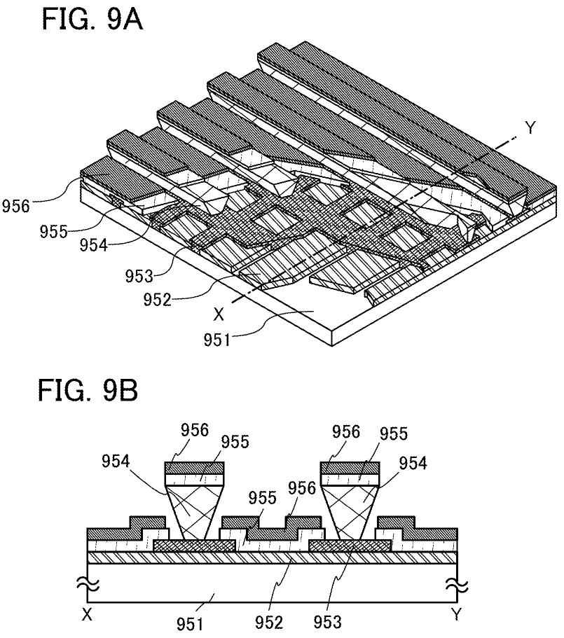

The active matrix light-emitting apparatus is described above, whereas a passive matrix light-emitting apparatus is described below. FIG. 9 illustrates a passive matrix light-emitting apparatus fabricated using the present invention. Note that FIG. 9A is a perspective view illustrating the light-emitting apparatus, and FIG. 9B is a cross-sectional view taken along X-Y in FIG. 9A. In FIG. 9, over a substrate 951, an EL layer 955 is provided between an electrode 952 and an electrode 956. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. That is, a cross section in the short side direction of the partition layer 954 has a trapezoidal shape, and the lower side (the side facing the same direction as the plane direction of the insulating layer 953 and touching the insulating layer 953) is shorter than the upper side (the side facing the same direction as the plane direction of the insulating layer 953, and not touching the insulating layer 953). The partition layer 954 thus provided can prevent defects in the light-emitting device due to static electricity or the like. The passive matrix light-emitting apparatus also uses the light-emitting device described in any one of Embodiment 1 to Embodiment 4; thus, the light-emitting apparatus can have favorable reliability or low power consumption.

In the light-emitting apparatus described above, many minute light-emitting devices arranged in a matrix can each be controlled; thus, the light-emitting apparatus can be suitably used as a display apparatus for displaying images.

This embodiment can be freely combined with any of the other embodiments.

### Embodiment 8

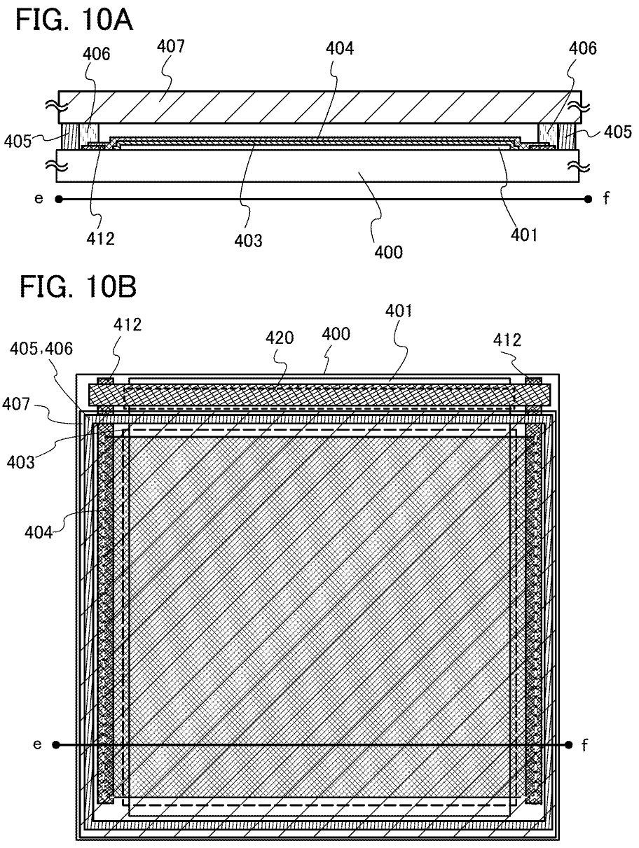

In this embodiment, an example in which the light-emitting device described in any one of Embodiment 1 to Embodiment 4 is used for a lighting device is described with reference to FIG. 10. FIG. 10B is a top view of the lighting device, and FIG. 10A is a cross-sectional view taken along e-f in FIG. 10B.

In the lighting device in this embodiment, a first electrode 401 is formed over a substrate 400 that is a support and has a light-transmitting property. The first electrode 401 corresponds to an electrode 101 in any one of Embodiment 1 to Embodiment 4. In the case where light emission is extracted from the first electrode 401 side, the first electrode 401 is formed using a material having a light-transmitting property.

A pad 412 for supplying a voltage to a second electrode 404 is formed over the substrate 400.

An EL layer 403 is formed over the first electrode 401. The EL layer 403 corresponds to the structure in which the layer 104, the unit 103, and the layer 105 are combined, the structure in which the layer 104, the unit 103, the intermediate layer 106, the unit 103_2, and the layer 105 are combined, or the like in any one of Embodiment 1 to Embodiment 4. Note that for these structures, the corresponding description can be referred to.

The second electrode 404 is formed to cover the EL layer 403. The second electrode 404 corresponds to an electrode 102 in any one of Embodiment 1 to Embodiment 4. In the case where light emission is extracted from the first electrode 401 side, the second electrode 404 is formed using a material having high reflectivity. The second electrode 404 is supplied with a voltage when connected to the pad 412.

As described above, the lighting device described in this embodiment includes a light-emitting device including the first electrode 401, the EL layer 403, and the second electrode 404. Since the light-emitting device is a light-emitting device with high emission efficiency, the lighting device in this embodiment can have low power consumption.

The substrate 400 provided with the light-emitting device having the above structure is fixed to a sealing substrate 407 with a sealant 405 and a sealant 406 and sealing is performed, whereby the lighting device is completed. It is possible to use only either the sealant 405 or the sealant 406. In addition, the inner sealant 406 (not shown in FIG. 10B) can be mixed with a desiccant, which enables moisture to be adsorbed, resulting in improved reliability.

When parts of the pad 412 and the first electrode 401 are provided to extend to the outside of the sealant 405 and the sealant 406, the extended parts can serve as external input terminals. An IC chip 420 mounted with a converter or the like may be provided over the external input terminals, for example.

The lighting device described in this embodiment includes the light-emitting device described in any one of Embodiment 1 to Embodiment 4 as an EL element; thus, the lighting device can have low power consumption.

### Embodiment 9

In this embodiment, examples of electronic devices each partly including the light-emitting device described in any one of Embodiment 1 to Embodiment 4 are described. The light-emitting device described in any one of Embodiment 1 to Embodiment 4 is a light-emitting device with favorable emission efficiency and low power consumption. As a result, the electronic devices described in this embodiment can be electronic devices each including a light-emitting portion with low power consumption.

Examples of the electronic device including the above light-emitting device include television devices (also referred to as TV or television receivers), monitors for computers and the like, digital cameras, digital video cameras, digital photo frames, cellular phones (also referred to as mobile phones or mobile phone devices), portable game machines, portable information terminals, audio playback devices, and large game machines such as pachinko machines. Specific examples of these electronic devices are described below.

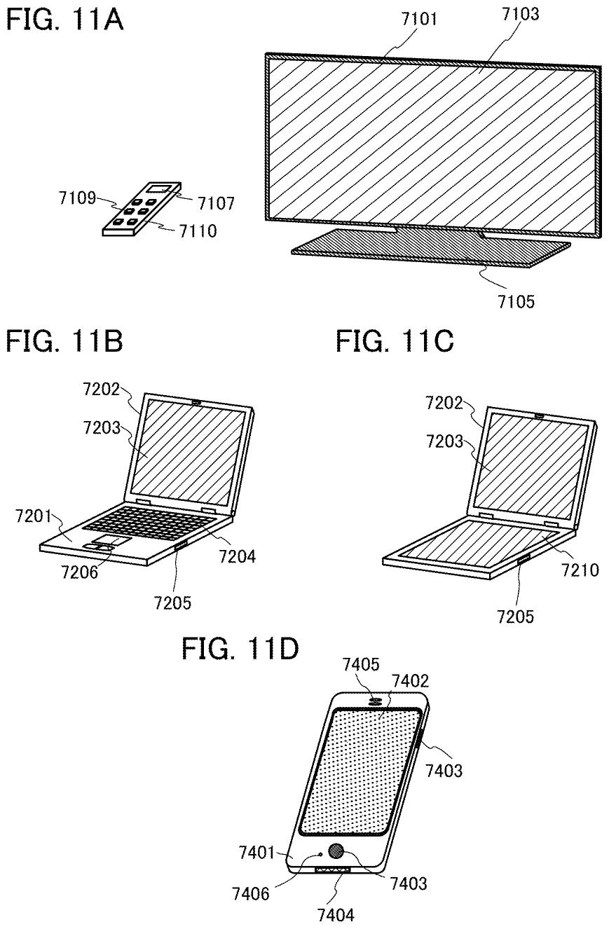

FIG. 11A illustrates an example of a television device. In the television device, a display portion 7103 is incorporated in a housing 7101. Here, the housing 7101 is supported by a stand 7105. Images can be displayed on the display portion 7103, and the light-emitting devices described in any one of Embodiment 1 to Embodiment 4 are arranged in a matrix in the display portion 7103.

The television device can be operated with an operation switch of the housing 7101 or a separate remote controller 7110. With operation keys 7109 of the remote controller 7110, channels and volume can be operated and images displayed on the display portion 7103 can be operated. Furthermore, the remote controller 7110 may be provided with a display portion 7107, on which information output from the remote controller 7110 may be displayed.

Note that the television device has a structure including a receiver, a modem, or the like. With the use of the receiver, a general television broadcast can be received, and moreover, when the television device is connected to a communication network with or without wires via the modem, one-way (from a sender to a receiver) or two-way (between a sender and a receiver or between receivers) information communication can be performed.

FIG. 11B shows a computer that includes a main body 7201, a housing 7202, a display portion 7203, a keyboard 7204, an external connection port 7205, a pointing device 7206, and the like. Note that this computer is fabricated using the light-emitting devices described in any one of Embodiment 1 to Embodiment 4 arranged in a matrix in the display portion 7203. The computer in FIG. 11B may be in a mode as illustrated in FIG. 11C. The computer in FIG. 11C is provided with a second display portion 7210 instead of the keyboard 7204 and the pointing device 7206. The second display portion 7210 is of a touch-panel type, and input can be performed by operating display for input displayed on the second display portion 7210 with a finger or a dedicated pen. The second display portion 7210 can also display images other than the display for input. The display portion 7203 may also be a touch panel. Connecting the two screens with a hinge can prevent troubles such as a crack in or damage to the screens caused when the computer is stored or carried.

FIG. 11D illustrates an example of a portable terminal. The portable terminal includes operation buttons 7403, an external connection port 7404, a speaker 7405, a microphone 7406, and the like in addition to a display portion 7402 incorporated in a housing 7401. Note that the portable terminal includes the display portion 7402 that is fabricated by arranging the light-emitting devices described in any one of Embodiment 1 to Embodiment 4 in a matrix.

The portable terminal illustrated in FIG. 11D can have a structure in which information can be input by touching the display portion 7402 with a finger or the like. In this case, operations such as making a call and creating an e-mail can be performed by touching the display portion 7402 with a finger or the like.

The display portion 7402 has mainly three screen modes. The first mode is a display mode mainly for displaying images. The second mode is an input mode mainly for inputting information such as text. The third mode is a display-and-input mode in which the two modes, the display mode and the input mode, are combined.

For example, in the case of making a call or creating an e-mail, the text input mode mainly for inputting text is selected for the display portion 7402 so that text displayed on the screen can be input. In this case, it is preferable to display a keyboard or number buttons on almost the entire screen of the display portion 7402.

When a detection device including a sensor such as a gyroscope sensor or an acceleration sensor for detecting inclination is provided inside the portable terminal, display on the screen of the display portion 7402 can be automatically changed by determining the orientation of the portable terminal (whether the portable terminal is placed vertically or horizontally).

The screen modes are switched by touching the display portion 7402 or operating the operation buttons 7403 of the housing 7401. Alternatively, the screen modes can be switched depending on the kind of images displayed on the display portion 7402. For example, when a signal of an image displayed on the display portion is moving image data, the screen mode is switched to the display mode. When the signal is text data, the screen mode is switched to the input mode.

Moreover, in the input mode, when input by touching the display portion 7402 is not performed for a certain period while a signal detected by an optical sensor in the display portion 7402 is sensed, the screen mode may be controlled so as to be switched from the input mode to the display mode.

The display portion 7402 can also function as an image sensor. For example, an image of a palm print, a fingerprint, or the like is taken when the display portion 7402 is touched with the palm or the finger, whereby personal authentication can be performed. Furthermore, by providing a backlight that emits near-infrared light or a sensing light source that emits near-infrared light in the display portion, an image of a finger vein, a palm vein, or the like can be taken.

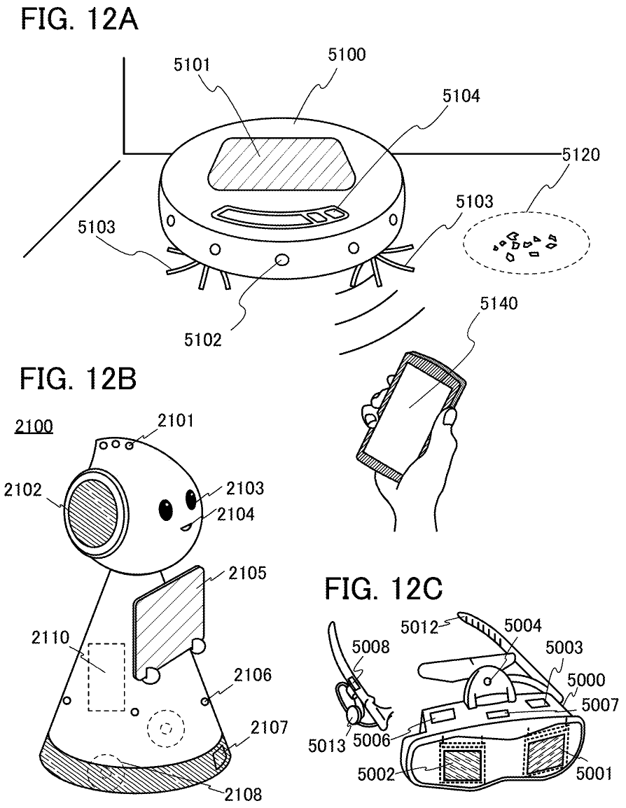

FIG. 12A is a schematic view showing an example of a cleaning robot.

A cleaning robot 5100 includes a display 5101 on its top surface, a plurality of cameras 5102 on its side surface, a brush 5103, and operation buttons 5104. Although not illustrated, the bottom surface of the cleaning robot 5100 is provided with a tire, an inlet, and the like. Furthermore, the cleaning robot 5100 includes various sensors such as an infrared sensor, an ultrasonic sensor, an acceleration sensor, a piezoelectric sensor, an optical sensor, and a gyroscope sensor. The cleaning robot 5100 has a wireless communication means.

The cleaning robot 5100 is self-propelled, senses dust 5120, and vacuums the dust through the inlet provided on the bottom surface.

The cleaning robot 5100 can determine whether there is an obstacle such as a wall, furniture, or a step by analyzing images taken by the cameras 5102. When an object that is likely to be caught in the brush 5103, such as a wire, is sensed by image analysis, the rotation of the brush 5103 can be stopped.

The display 5101 can display the remaining capacity of a battery, the amount of vacuumed dust, or the like. The display 5101 may display a path on which the cleaning robot 5100 has run. The display 5101 may be a touch panel, and the operation buttons 5104 may be provided on the display 5101.

The cleaning robot 5100 can communicate with a portable electronic device 5140 such as a smartphone. Images taken by the cameras 5102 can be displayed on the portable electronic device 5140. Accordingly, an owner of the cleaning robot 5100 can monitor his/her room even when the owner is not at home. The owner can also check the display on the display 5101 by the portable electronic device 5140 such as a smartphone.

The light-emitting apparatus of one embodiment of the present invention can be used for the display 5101.

A robot 2100 illustrated in FIG. 12B includes an arithmetic device 2110, an illuminance sensor 2101, a microphone 2102, an upper camera 2103, a speaker 2104, a display 2105, a lower camera 2106, an obstacle sensor 2107, and a moving mechanism 2108.

The microphone 2102 has a function of sensing a speaking voice of a user, an environmental sound, and the like. The speaker 2104 has a function of outputting sound. The robot 2100 can communicate with a user by using the microphone 2102 and the speaker 2104.

The display 2105 has a function of displaying various kinds of information. The robot 2100 can display information desired by a user on the display 2105. The display 2105 may be provided with a touch panel. Moreover, the display 2105 may be a detachable information terminal, in which case charging and data communication can be performed when the display 2105 is set at the home position of the robot 2100.

The upper camera 2103 and the lower camera 2106 each have a function of taking an image of the surroundings of the robot 2100. The obstacle sensor 2107 can detect an obstacle in the direction where the robot 2100 advances with the moving mechanism 2108. The robot 2100 can move safely by recognizing the surroundings with the upper camera 2103, the lower camera 2106, and the obstacle sensor 2107. The light-emitting apparatus of one embodiment of the present invention can be used for the display 2105.

FIG. 12C is a diagram illustrating an example of a goggle-type display. The goggle-type display includes, for example, a housing 5000, a display portion 5001, a speaker 5003, an LED lamp 5004, operation keys (including a power switch or an operation switch), a connection terminal 5006, a sensor 5007 (a sensor having a function of measuring force, displacement, position, speed, acceleration, angular velocity, rotational frequency, distance, light, liquid, magnetism, temperature, a chemical substance, sound, time, hardness, an electric field, current, voltage, power, radiation, flow rate, humidity, gradient, oscillation, odor, or infrared rays), a microphone 5008, a display portion 5002, a support 5012, and an earphone 5013.

The light-emitting apparatus of one embodiment of the present invention can be used for the display portion 5001 and the display portion 5002.

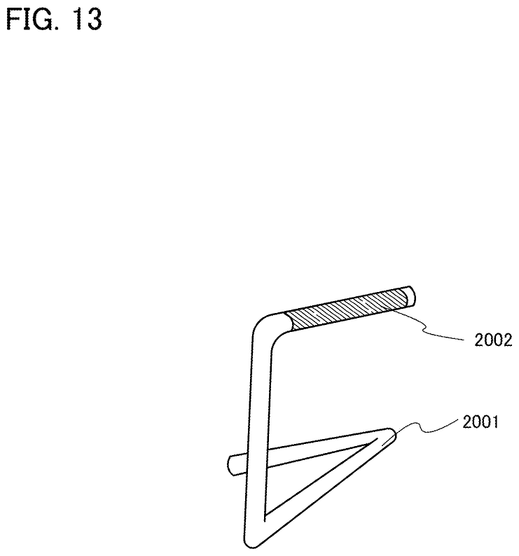

FIG. 13 shows an example where the light-emitting device described in any one of Embodiment 1 to Embodiment 4 is used for a table lamp which is a lighting device. The table lamp illustrated in FIG. 13 includes a housing 2001 and a light source 2002, and the lighting device described in Embodiment 8 may be used for the light source 2002.

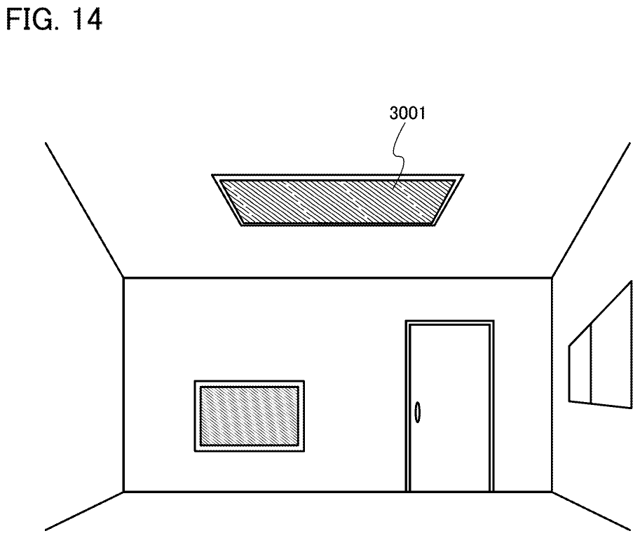

FIG. 14 shows an example where the light-emitting device described in any one of Embodiment 1 to Embodiment 4 is used for an indoor lighting device 3001. Since the light-emitting device described in any one of Embodiment 1 to Embodiment 4 is a light-emitting device with high emission efficiency, the lighting device can have low power consumption. In addition, the light-emitting device described in any one of Embodiment 1 to Embodiment 4 can have a larger area, and thus can be used for a large-area lighting device. Furthermore, the light-emitting device described in any one of Embodiment 1 to Embodiment 4 is thin, and thus can be used for a lighting device having a reduced thickness.

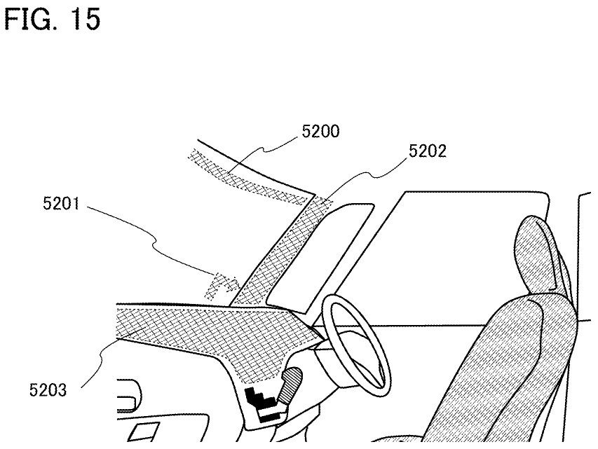

The light-emitting device described in any one of Embodiment 1 to Embodiment 4 can also be incorporated in a windshield or a dashboard of an automobile. FIG. 15 illustrates one mode in which the light-emitting device described in any one of Embodiment 1 to Embodiment 4 is used for a windshield or a dashboard of an automobile. A display region 5200 to a display region 5203 are each a display region provided using the light-emitting device described in any one of Embodiment 1 to Embodiment 4.

The display region 5200 and the display region 5201 are display apparatuses provided in the automobile windshield, in which the light-emitting devices described in any one of Embodiment 1 to Embodiment 4 are incorporated. When the light-emitting devices described in any one of Embodiment 1 to Embodiment 4 are fabricated using electrodes having light-transmitting properties as a first electrode and a second electrode, what is called see-through display apparatuses, through which the opposite side can be seen, can be obtained. Such see-through display apparatuses can be provided even in the automobile windshield without hindering the view. Note that in the case where a driving transistor or the like is provided, a transistor having a light-transmitting property, such as an organic transistor formed using an organic semiconductor material or a transistor formed using an oxide semiconductor, is preferably used.

The display region 5202 is a display apparatus provided in a pillar portion, in which the light-emitting devices described in any one of Embodiment 1 to Embodiment 4 are incorporated. The display region 5202 can compensate for the view hindered by the pillar by displaying an image taken by an image capturing means provided in the car body. Similarly, the display region 5203 provided in the dashboard portion can compensate for the view hindered by the car body by displaying an image taken by an image capturing means provided on the outside of the automobile; thus, blind areas can be eliminated to enhance the safety. Images that compensate for the areas that a driver cannot see enable the driver to ensure safety easily and comfortably.

The display region 5203 can provide a variety of information by displaying navigation information, speed, revolutions, a mileage, a fuel level, a gearshift state, air-condition setting, and the like. The content or layout of the display can be changed freely in accordance with the preference of a user. Note that such information can also be displayed on the display region 5200 to the display region 5202. The display region 5200 to the display region 5203 can also be used as lighting devices.

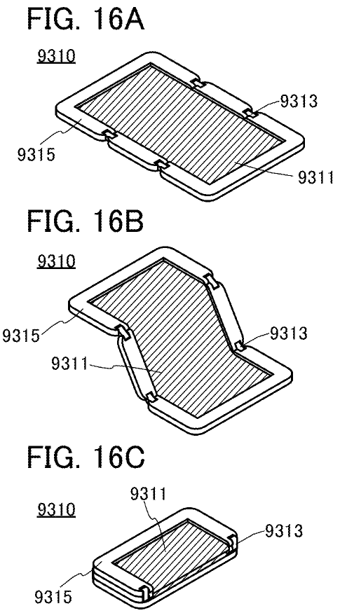

FIG. 16A to FIG. 16C illustrate a foldable portable information terminal 9310. FIG. 16A illustrates the portable information terminal 9310 that is opened. FIG. 16B illustrates the portable information terminal 9310 that is in the state of being changed from one of an opened state and a folded state to the other. FIG. 16C illustrates the portable information terminal 9310 that is folded. The portable information terminal 9310 is excellent in portability when folded, and is excellent in display browsability when opened because of a seamless large display region.

A display panel 9311 is supported by three housings 9315 joined together by hinges 9313. Note that the display panel 9311 may be a touch panel (an input/output device) that includes a touch sensor (an input device). By folding the display panel 9311 at the hinges 9313 between two housings 9315, the portable information terminal 9310 can be reversibly changed in shape from the opened state to the folded state. The light-emitting apparatus of one embodiment of the present invention can be used for the display panel 9311.

Note that the structures described in this embodiment can be combined with any of the structures described in Embodiment 1 to Embodiment 4 as appropriate.

As described above, the application range of the light-emitting apparatus that includes the light-emitting device described in any one of Embodiment 1 to Embodiment 4 is wide, so that this light-emitting apparatus can be applied to electronic devices in a variety of fields. With the use of the light-emitting device described in any one of Embodiment 1 to Embodiment 4, an electronic device with low power consumption can be obtained.

Note that this embodiment can be combined with any of the other embodiments described in this specification as appropriate.

### Example

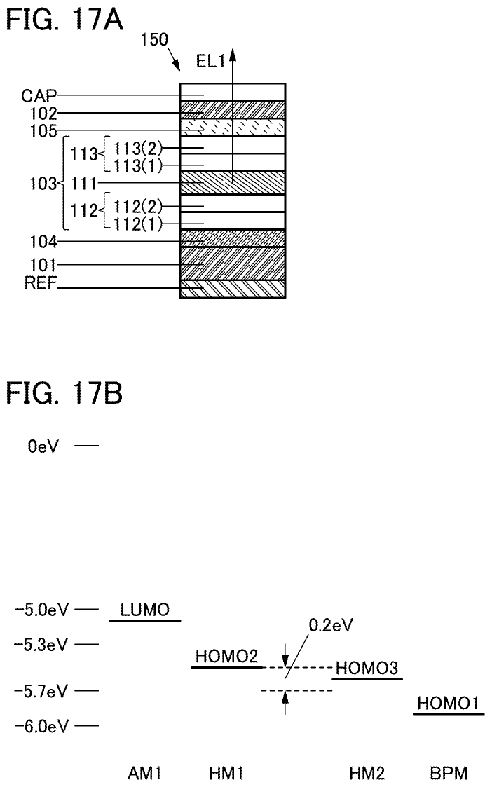

In this example, a light-emitting device 1 and a light-emitting device 2 of embodiments of the present invention are described with reference to FIG. 17 to FIG. 24.

FIG. 17 is a diagram illustrating the structure of the light-emitting device 150.

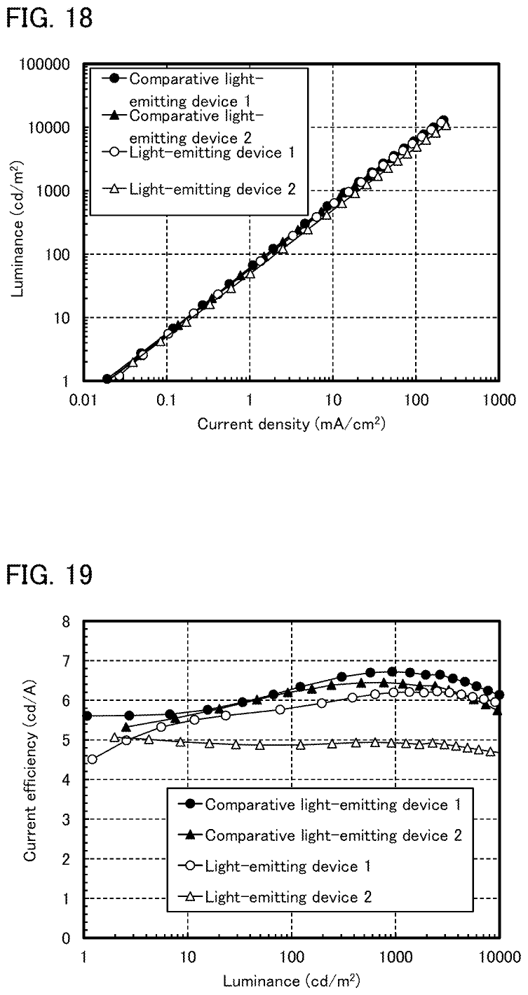

FIG. 18 is a diagram illustrating the current density-luminance characteristics of the light-emitting device 1 and the light-emitting device 2.

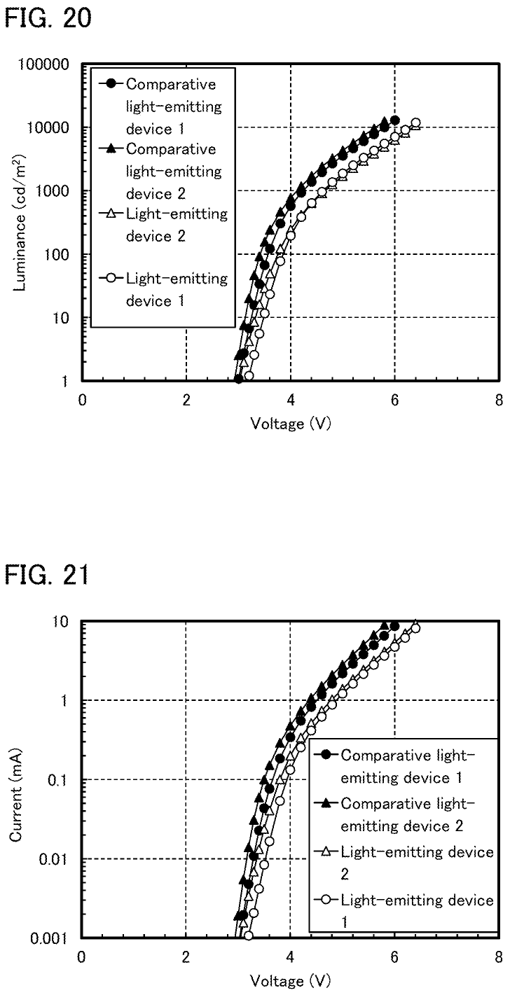

FIG. 19 is a diagram illustrating the luminance-current efficiency characteristics of the light-emitting device 1 and the light-emitting device 2.

FIG. 20 is a diagram illustrating the voltage-luminance characteristics of the light-emitting device 1 and the light-emitting device 2.

FIG. 21 is a diagram illustrating the voltage-current characteristics of the light-emitting device 1 and the light-emitting device 2.

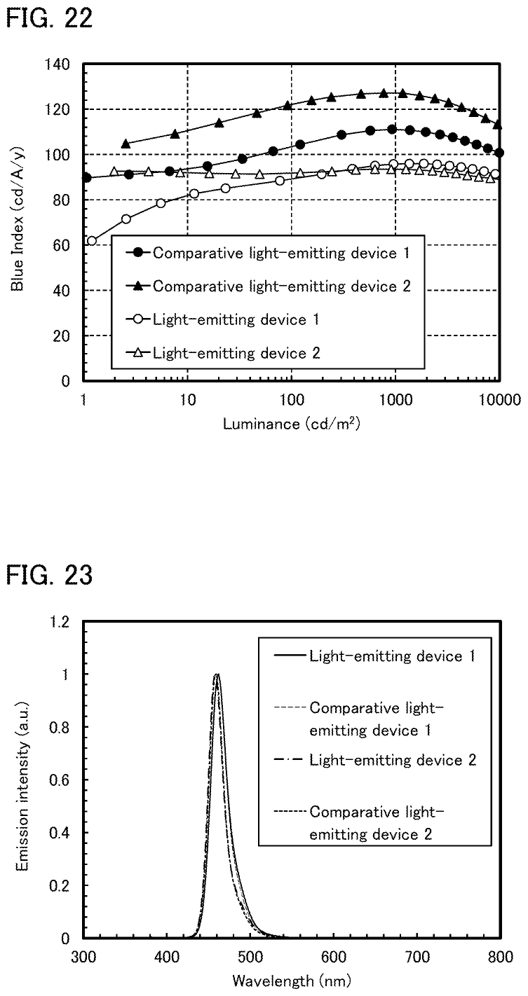

FIG. 22 is a diagram illustrating the luminance-blue index characteristics of the light-emitting device 1 and the light-emitting device 2.

FIG. 23 is a diagram illustrating the emission spectra of the light-emitting device 1 and the light-emitting device 2 each emitting light at a luminance of 1000 cd/m².

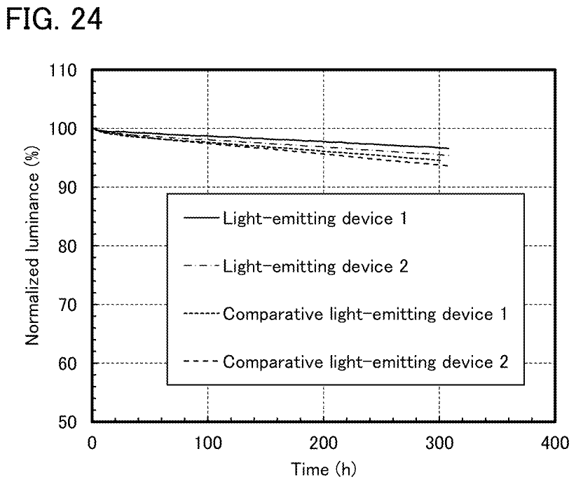

FIG. 24 is a diagram illustrating a temporal change in the normalized luminance of the light-emitting device 1 and the light-emitting device 2 each emitting light at a constant current density of 50 mA/cm²

### <Light-Emitting Device 1>

The fabricated light-emitting device 1, which is described in this example, has a structure similar to that of the light-emitting device 150 (see FIG. 17). The light-emitting device 150 includes the electrode 101, the electrode 102, the unit 103, and the layer 104. The unit 103 is held between the electrode 101 and the electrode 102, and the unit 103 includes the layer 111, the layer 112, and the layer 113. The layer 111 is held between the layer 112 and the layer 113, and the layer 111 contains a light-emitting material. The layer 113 is held between the layer 111 and the electrode 102, and the layer 113 contains the organic compound BPM. The organic compound BPM has a π-electron deficient heteroaromatic ring skeleton and a π-electron rich heteroaromatic ring skeleton. Note that the layer 113 includes a layer 113(1) and a layer 113(2), and the layer 112 includes a layer 112(1) and a layer 112(2). The layer 104 is held between the electrode 551 and the unit 103, is in contact with the electrode 101, and contains the organic compound HM1 and the organic compound AM1. The organic compound AM1 has an electron-accepting property with respect to the organic compound HM1, and the layer 104 has a resistivity higher than or equal to 1×10⁴ [Ω·cm] and lower than or equal to 1×10⁷ [Ω·cm].

### <<Structure of Light-Emitting Device 1>>

Table 1 shows the structure of the light-emitting device 1. The structural formulae of the materials used in the light-emitting devices described in this example are shown below. Note that in the tables in this example, subscript and superscript characters are written in ordinary size for convenience. For example, a subscript character in an abbreviation or a superscript character in a unit are written in ordinary size in the tables. The corresponding description in the specification gives an accurate reading of such notations in the tables.

TABLE 1

| | Reference | | Composition | Thickness/ |
|---|---|---|---|---|
| Component | numeral | Material | ratio | nm |
| Layer | CAP | DBT3P-II | | 80 |
| Electrode | 102 | Ag:Mg | 1:0.1 | 15 |
| Layer | 105 | LiF | | 1 |
| Layer | 113(2) | NBPhen | | 10 |
| Layer | 113(1) | 2mpPCBPDBq | | 20 |
| Layer | 111 | αN-βNPAnth:3,10PCA2Nbf(IV)-02 | 1:0.015 | 25 |
| Layer | 112(2) | PCzN2 | | 10 |
| Layer | 112(1) | BBABnf | | 20 |
| Layer | 104 | BBABnf:OCHD-003 | 1:0.1 | 10 |
| Electrode | 101 | ITSO | | 85 |
| Reflective film | REF | Ag | | 100 |

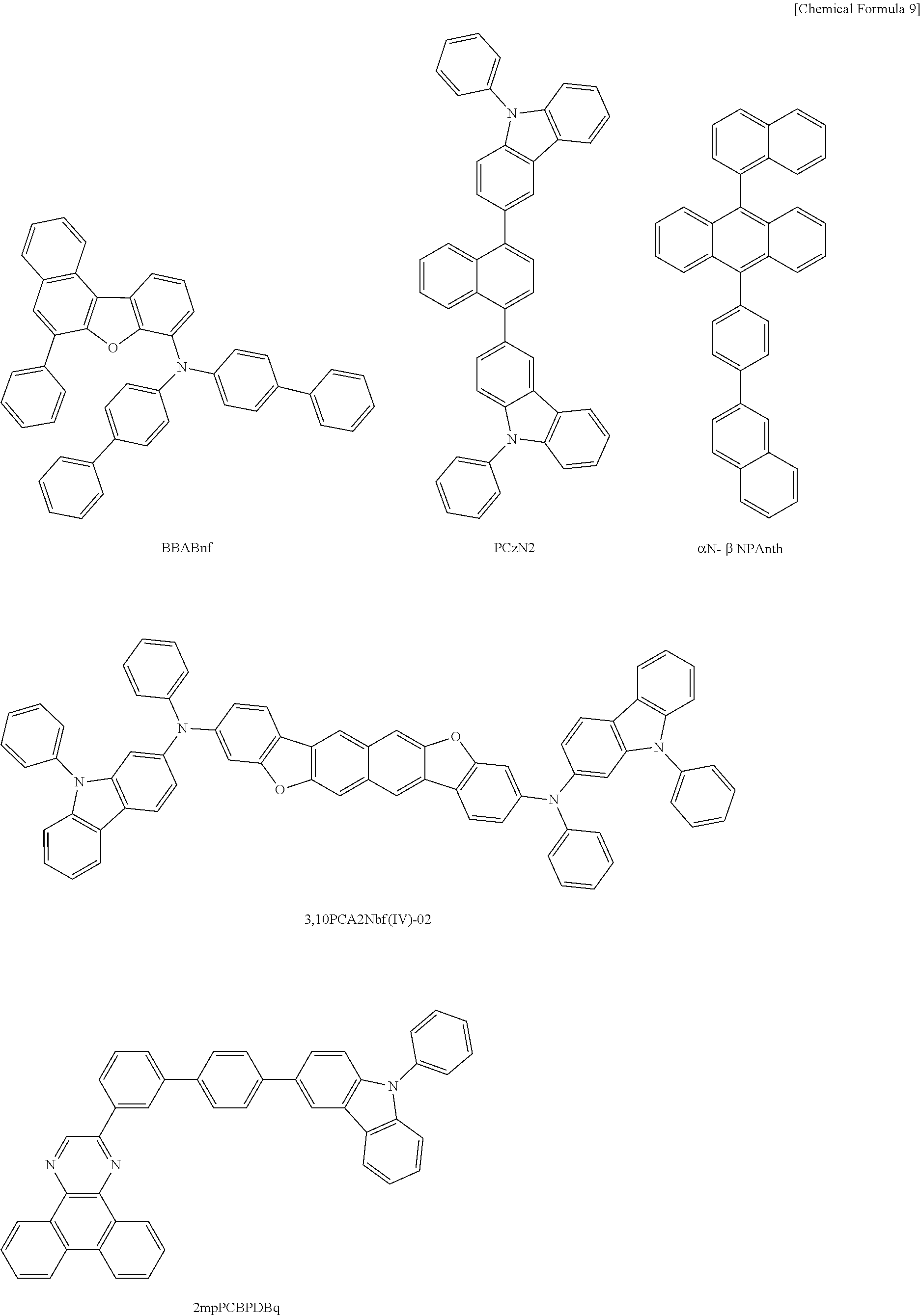

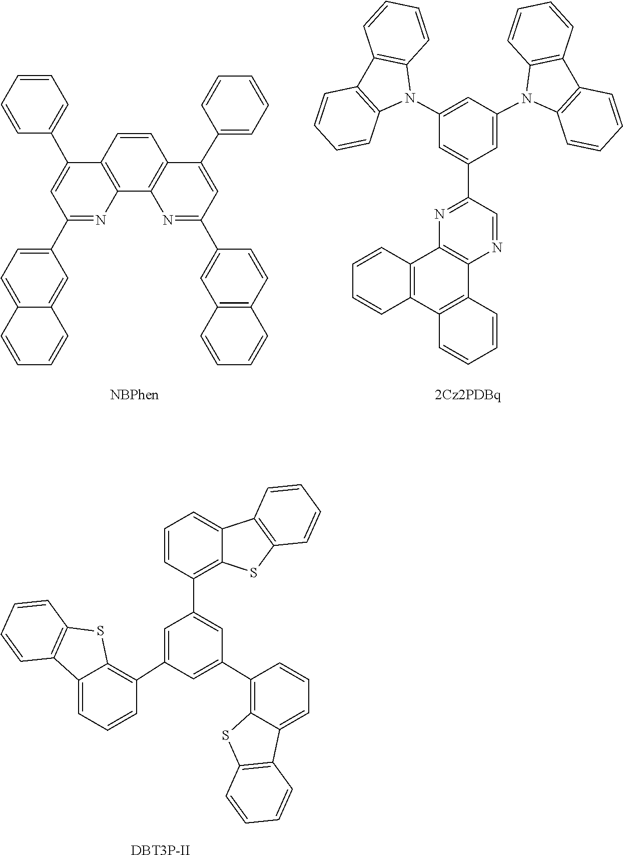 [SMILES: c1ccc(-c2cc(-c3ccc4ccccc4c3)nc3c2ccc2c(-c4ccccc4)cc(-c4ccc5ccccc5c4)nc23)cc1 ; c1ccc2c(c1)c1ccccc1c1nc(-c3cc(-n4c5ccccc5c5ccccc54)cc(-n4c5ccccc5c5ccccc54)c3)cnc21 ; c1ccc2c(c1)sc1c(-c3cc(-c4cccc5c4sc4ccccc45)cc(-c4cccc5c4sc4ccccc45)c3)cccc12 ; "DBT3P-II"]

### <<Fabrication Method of Light-Emitting Device 1>>

The light-emitting device 1 described in this example was fabricated using a method including the following steps.

### [First Step]

In the first step, a reflective film REF was formed. Specifically, the reflective film REF was formed by a sputtering method using silver (Ag) as a target.

The reflective film REF contains Ag and has a thickness of 100 nm.

### [Second Step]

In the second step, the electrode 101 was formed over the reflective film REF. Specifically, the electrode 101 was formed by a sputtering method using indium oxide-tin oxide containing silicon or silicon oxide (abbreviation: ITSO) as a target.

The electrode 101 contains ITSO and has a thickness of 85 nm and an area of 4 mm² (2 mm×2 mm).

Next, a substrate over which the electrode 101 was formed was washed with water, baked at 200° C. for 1 hour, and then subjected to UV ozone treatment for 370 seconds. After that, the substrate was transferred into a vacuum evaporation apparatus where the pressure was reduced to approximately 10-4 Pa, and vacuum baking was performed at 170° C. for 30 minutes in a heating chamber of the vacuum evaporation apparatus. Then, the substrate was cooled down for approximately 30 minutes.

### [Third Step]

In the third step, the layer 104 was formed over the electrode 101. Specifically, the materials were deposited by co-evaporation using a resistance-heating method.

Note that the layer 104 contains N,N-bis(4-biphenyl)-6-phenylbenzo[b]naphtho[1,2-d]furan-8-amine (abbreviation: BBABnf) and an electron-accepting material (abbreviation: OCHD-003) at BBABnf: OCHD-003=1:0.1 (weight ratio) and has a thickness of 10 nm. The HOMO level of BBABnf is-5.6 eV (see FIG. 17B). The electron-accepting material OCHD-003 contains fluorine, and has a molecular weight of 672.

### [Fourth Step]

In the fourth step, the layer 112(1) was formed over the layer 104. Specifically, the material was deposited by evaporation using a resistance-heating method.

The layer 112(1) contains BBABnf and has a thickness of 20 nm.

### [Fifth Step]

In the fifth step, the layer 112(2) was formed over the layer 112(1). Specifically, the material was deposited by evaporation using a resistance-heating method.

Note that the layer 112(2) contains 3,3′-(naphthalene-1,4-diyl)bis(9-phenyl-9H-carbazole) (abbreviation: PCzN2) and has a thickness of 10 nm.

### [Sixth Step]

In the sixth step, the layer 111 was formed over the layer 112(2). Specifically, the materials were deposited by co-evaporation using a resistance-heating method.

Note that the layer 111 contains 9-(1-naphthyl)-10-[4-(2-naphthyl)phenyl]anthracene (abbreviation: αN-BNP Anth) and 3,10-bis[N-(9-phenyl-9H-carbazol-2-yl)-N-phenylamino]naphtho[2,3-b;6,7-b′]bisbenzofuran (abbreviation: 3,10PCA2Nbf (IV)-02) at αN-BNPAnth: 3,10PCA2Nbf (IV)-02=1:0.015 (weight ratio) and has a thickness of 25 nm.

### [Seventh Step]

In the seventh step, the layer 113(1) was formed over the layer 111. Specifically, the material was deposited by evaporation using a resistance-heating method.

The layer 113(1) contains 2-[4′-(9-phenyl-9H-carbazol-3-yl)-3,1′-biphenyl-1-yl]dibenzo[f,h]quinoxaline (abbreviation: 2mpPCBPDBq) and has a thickness of 20 nm.

2mpPCBPDBq has a carbazole skeleton. Furthermore, 2mpPCBPDBq has a HOMO level higher than or equal to −6.0 eV and lower than or equal to −5.6 eV (see FIG. 17B). Accordingly, transfer of holes from the layer 111 toward the layer 113(1) is facilitated. Furthermore, a region contributing to light emission near the layer 111 can be moderately expanded.

### [Eighth Step]

In the eighth step, the layer 113(2) was formed over the layer 113(1). Specifically, the material was deposited by evaporation using a resistance-heating method.

The layer 113(2) contains 2,9-di(2-naphthyl)-4,7-diphenyl-1,10-phenanthroline (abbreviation: NBPhen) and has a thickness of 10 nm.

### [Ninth Step]

In the ninth step, the layer 105 was formed over the layer 113(2). Specifically, the material was deposited by evaporation using a resistance-heating method.

The layer 105 contains LiF and has a thickness of 1 nm.

### [Tenth Step]

In the tenth step, the electrode 102 was formed over the layer 105. Specifically, the materials were deposited by co-evaporation using a resistance-heating method.

The electrode 102 contains Ag and Mg at Ag:Mg=1:0.1 (volume ratio) and has a thickness of 15 nm.

### [Eleventh Step]

In the eleventh step, a layer CAP was formed over the electrode 102. Specifically, the material was deposited by evaporation using a resistance-heating method.

The layer CAP contains 4,4′,4″-(benzene-1,3,5-triyl)tri (dibenzothiophene) (abbreviation: DBT3P-II) and has a thickness of 80 nm.

### <<Operation Characteristics of Light-Emitting Device 1>>

When supplied with electric power, the light-emitting device 1 emitted the light EL 1 (see FIG. 17). The operation characteristics of the light-emitting device 1 were measured at room temperature (see FIG. 18 to FIG. 24). The luminance, CIE chromaticity, and emission spectrum were measured using a spectroradiometer (SR-ULIR, manufactured by TOPCON TECHNOHOUSE CORPORATION). Table 2 also shows the characteristics of the other light-emitting devices, whose structures will be described later.

Table 2 shows the main initial characteristics and the reliability test results of the fabricated light-emitting devices emitting light at a luminance of approximately 1000 cd/m².

Note that the blue index (BI) is one of the indicators of characteristics of a blue-light-emitting device, and is a value obtained by dividing current efficiency (cd/A) by chromaticity y. In general, blue light with high color purity is useful in expressing a wide color gamut. In addition, blue light with higher color purity tends to have lower chromaticity y. Thus, a value obtained by dividing current efficiency (cd/A) by chromaticity y is the indicator of usefulness of a blue-light-emitting device. In other words, a blue-light-emitting device with a large BI is suitable for providing a display apparatus having a wide color gamut and high efficiency.

The reliability of the light-emitting devices emitting light at a constant current density (50 mA/cm²) was evaluated (see FIG. 24). For the evaluation, the proportion of the luminance after 310 hours elapsed with respect to the initial luminance was used.

TABLE 2

| | | | Current | | | Current | | 310 hr |
|---|---|---|---|---|---|---|---|---|
| | Voltage | Current | density | | | efficiency | B.I. value | @50 mA/cm2 |
| | (V) | (mA) | (mA/cm2) | Chromaticity x | Chromaticity y | (cd/A) | (cd/A/y) | (%) |
| Light-emitting | 4.6 | 0.62 | 15.4 | 0.13 | 0.06 | 6.2 | 95.8 | 97% |
| device 1 | | | | | | | | |
| Light-emitting | 4.6 | 0.74 | 18.5 | 0.14 | 0.05 | 4.9 | 93.5 | 95% |
| device 2 | | | | | | | | |
| Comparative light- | 4.2 | 0.55 | 13.8 | 0.14 | 0.06 | 6.7 | 111.1 | 94% |
| emitting device 1 | | | | | | | | |
| Comparative light- | 4.2 | 0.73 | 18.3 | 0.14 | 0.05 | 6.4 | 127.1 | 94% |
| emitting device 2 | | | | | | | | |

The light-emitting device 1 was found to have favorable characteristics. For example, the light-emitting device 1 had higher reliability than a comparative light-emitting device 1 and a comparative light-emitting device 2. 2mpPCBPDBq has a carbazole skeleton with a hole-transport property and has a HOMO level of −5.81 eV. Note that αN-βNP Anth used in the layer 111 has a HOMO level of −5.85 eV. Holes are easily transferred from the layer 111 formed using αN-βNPAnth to the layer 113(1) formed using 2mpPCBPDBq since this transfer is from the deeper HOMO level to the shallower HOMO level. In addition, accumulation of holes between the layer 111 and the layer 113(1) can be reduced.

### <Light-Emitting Device 2>

The fabricated light-emitting device 2, which is described in this example, has a structure similar to that of the light-emitting device 150 (see FIG. 17).

### <<Structure of Light-Emitting Device 2>>

The structure of the light-emitting device 2 is different from that of the light-emitting device 1 in the layer 113(1). Specifically, the light-emitting device 2 is different from the light-emitting device 1 in including 3-[3,5-di(carbazol-9-yl)phenyl]phenanthro[9,10-b]pyrazine (abbreviation: 2Cz2PDBq) instead of 2mpPCBPDBq.

### <<Fabrication Method of Light-Emitting Device 2>>

The light-emitting device 2 described in this example was fabricated using a method including the following steps.

The fabrication method of the light-emitting device 2 is different from that of the light-emitting device 1 in using 2Cz2PDBq instead of 2mpPCBPDBq in the step of forming the layer 113(1). Different portions are described in detail here, and the above description is referred to for portions formed by a similar method.

### [Seventh Step]

In the seventh step, the layer 113(1) was formed over the layer 111. Specifically, the material was deposited by evaporation using a resistance-heating method.

Note that the layer 113(1) contains 2Cz2PDBq and has a thickness of 20 nm.

### <<Operation Characteristics of Light-Emitting Device 2>>

When supplied with electric power, the light-emitting device 2 emitted the light EL1 (see FIG. 17). The operation characteristics of the light-emitting device 2 were measured at room temperature (see FIG. 18 to FIG. 24). The luminance, CIE chromaticity, and emission spectrum were measured using a spectroradiometer (SR-ULIR, manufactured by TOPCON TECHNOHOUSE CORPORATION).

Table 2 shows the main initial characteristics and the reliability test results of the fabricated light-emitting devices emitting light at a luminance of approximately 1000 cd/m². The light-emitting device 2 was found to have favorable characteristics. For example, the light-emitting device 2 had higher reliability than the comparative light-emitting device 1 and the comparative light-emitting device 2.

### Reference Example

The fabricated comparative light-emitting device 1 described in this reference example has a structure similar to that of the light-emitting device 150 (see FIG. 17).

### <<Structure of Comparative Light-Emitting Device 1>>

The structure of the comparative light-emitting device 1 is different from that of the light-emitting device 1 in the layer 113(1) and the layer 113(2).

The comparative light-emitting device 1 is different from the light-emitting device 1 in that the layer 113(1) has a thickness of 10 nm, not 20 nm. The comparative light-emitting device 1 is different from the light-emitting device 1 in that the layer 113(1) contains 2-[3-(3′-dibenzothiophen-4-yl) biphenyl]dibenzo[f,h]quinoxaline (abbreviation: 2mDBTBPDBq-II) instead of 2mpPCBPDBq. 2mpPCBPDBq has a carbazole skeleton with a hole-transport property and has a HOMO level of −5.81 eV. Meanwhile, 2mDBTBPDBq-II has a thiophene skeleton with a hole-transport property but has a HOMO level of −6.22 eV. Note that αN-βNP Anth used in the layer 111 has a HOMO level of −5.85 eV.

Hole transfer from the layer 111 formed using αN-βNPAnth to the layer 113(1) formed using 2mDBTBPDBq-II is hole transfer from the shallower HOMO level to the deeper HOMO level and is more difficult than hole transfer from the layer 111 formed using αN-βNP Anth to the layer 113(1) formed using 2mpPCBPDBq.

The comparative light-emitting device 1 is different from the light-emitting device 1 in that the layer 113(2) has a thickness of 20 nm, not 10 nm.

The structural formula of the material used in the comparative light-emitting device 1 described in this reference example is shown below.

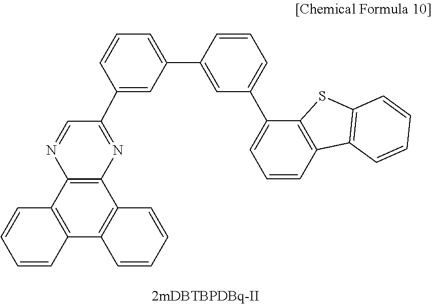 [SMILES: c1cc(-c2cccc(-c3cccc4c3sc3ccccc34)c2)cc(-c2cnc3c4ccccc4c4ccccc4c3n2)c1]

### <<Fabrication Method of Comparative Light-Emitting Device 1>>

The comparative light-emitting device 1 described in this reference example was fabricated using a method including the following steps.

The fabrication method of the comparative light-emitting device 1 is different from that of the light-emitting device 1 in that in the step of forming the layer 113(1), its thickness was set to 10 nm, not 20 nm, and 2mDBTBPDBq-II was used instead of 2mpPCBPDBq. The fabrication method of the comparative light-emitting device 1 is different from that of the light-emitting device 1 in that in the step of forming the layer 113(2), its thickness was set to 20 nm, not 10 nm. Different portions are described in detail here, and the above description is referred to for portions formed by a similar method.

### [Seventh Step]

In the seventh step, the layer 113(1) was formed over the layer 111. Specifically, the material was deposited by evaporation using a resistance-heating method.

Note that the layer 113(1) contains 2mDBTBPDBq-II and has a thickness of 10 nm.

### [Eighth Step]

In the eighth step, the layer 113(2) was formed over the layer 113(1). Specifically, the material was deposited by evaporation using a resistance-heating method.

Note that the layer 113(2) contains NBPhen and has a thickness of 20 nm.

### <<Structure of Comparative Light-Emitting Device 2>>

The structure of the comparative light-emitting device 2 is different from that of the light-emitting device 1 in the layer 113(1) and the layer 113(2).

The comparative light-emitting device 2 is different from the light-emitting device 1 in that the layer 113(1) contains 2-{4-[9,10-di(2-naphthyl)-2-anthryl]phenyl}-1-phenyl-1H-benzimidazole (abbreviation: ZADN) and 8-quinolinolato-lithium (abbreviation: Liq) at a weight ratio of 1:1, instead of 2mpPCBPDBq. ZADN has an imidazole skeleton that is a π-electron deficient heteroaromatic ring skeleton but does not have a π-electron rich heteroaromatic ring skeleton.

The structural formulae of the materials used in the comparative light-emitting device 2 described in this reference example are shown below.

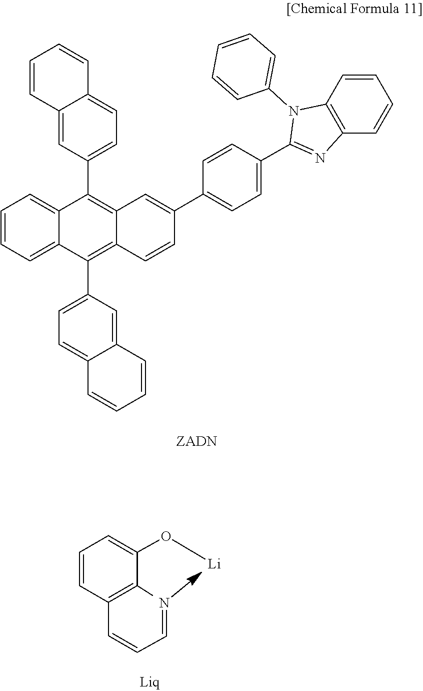

### <<Fabrication Method of Comparative Light-Emitting Device 2>>

The comparative light-emitting device 2 described in this reference example was fabricated using a method including the following steps.

The fabrication method of the comparative light-emitting device 2 is different from that of the light-emitting device 1 in that a material obtained by mixing ZADN and Liq at a weight ratio of 1:1 was used instead of 2mpPCBPDBq in the step of forming the layer 113(1). Different portions are described in detail here, and the above description is referred to for portions formed by a similar method.

### [Seventh Step]

In the seventh step, the layer 113(1) was formed over the layer 111. Specifically, the material was deposited by evaporation using a resistance-heating method.

Note that the layer 113(1) contains ZADN and Liq at ZADN:Liq=1:1 (weight ratio) and has a thickness of 20 nm.

### REFERENCE NUMERALS

AM1: organic compound, BPM: organic compound, EL1: light, EL1_2: light, EL2: light, HM1: organic compound, HM2: organic compound, HOMO1: HOMO level, HOMO2: HOMO level, HOMO3: HOMO level, 101: electrode, 102: electrode, 103: unit, 103_2: unit, 103S: unit, 103X: unit, 103Y: unit, 104: layer, 104X: layer, 104XY: gap, 104Y: layer, 105: layer, 105_2: layer, 106: intermediate layer, 106_1: layer, 106_2: layer, 111: layer, 111X: layer, 111Y: layer, 112: layer, 112X: layer, 112Y: layer, 113: layer, 113X: layer, 113Y: layer, 114N: layer, 114P: layer, 114S: layer, 150: light-emitting device, 400: substrate, 401: electrode, 403: EL layer, 404: electrode, 405: sealant, 406: sealant, 407: sealing substrate, 412: pad, 420: IC chip, 521: insulating film, 528: insulating film, 550: light-emitting device, 550S: optical functional device, 550X: light-emitting device, 550Y: light-emitting device, 551: electrode, 551S: electrode, 551X: electrode, 551XY: gap, 551Y: electrode, 552: electrode, 573: insulating film, 573A: insulating film, 573B: insulating film, 601: source line driver circuit, 602: pixel portion, 603: gate line driver circuit, 604: sealing substrate, 605: sealant, 607: space, 608: wiring, 610: element substrate, 611: switching FET, 612: current control FET, 613: electrode, 614: insulator, 616: EL layer, 617: electrode, 618: light-emitting device, 623: FET, 700: functional panel, 951: substrate, 952: electrode, 953: insulating layer, 954: partition layer, 955: EL layer, 956: electrode, 1001: substrate, 1002: base insulating film, 1003: gate insulating film, 1006: gate electrode, 1007: gate electrode, 1008: gate electrode, 1020: interlayer insulating film, 1021: interlayer insulating film, 1022: electrode, 1024B: electrode, 1024G: electrode, 1024R: electrode, 1024W: electrode, 1025: partition, 1028: EL layer, 1029: electrode, 1031: sealing substrate, 1032: sealant, 1033: base material, 1034B: coloring layer, 1034G: coloring layer, 1034R: coloring layer, 1035: black matrix, 1036: overcoat layer, 1037: interlayer insulating film, 1040: pixel portion, 1041: driver circuit portion, 1042: peripheral portion, 2001: housing, 2002: light source, 2100: robot, 2101: illuminance sensor, 2102: microphone, 2103: upper camera, 2104: speaker, 2105: display, 2106: lower camera, 2107: obstacle sensor, 2108: moving mechanism, 2110: arithmetic device, 3001: lighting device, 5000: housing, 5001: display portion, 5002: display portion, 5003: speaker, 5004: LED lamp, 5006: connection terminal, 5007: sensor, 5008: microphone, 5012: support, 5013: earphone, 5100: cleaning robot, 5101: display, 5102: camera, 5103: brush, 5104: operation button, 5120: dust, 5140: portable electronic device, 5200: display region, 5201: display region, 5202: display region, 5203: display region, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7210: display portion, 7401: housing, 7402: display portion, 7403: operation button, 7404: external connection port, 7405: speaker, 7406: microphone, 9310: portable information terminal, 9311: display panel, 9313: hinge, 9315: housing

1. A light-emitting device comprising:
a first electrode;
a second electrode;
a first unit between the first electrode and the second electrode; and
a first layer between the first electrode and the first unit,
wherein the first unit comprises a third layer, a second layer between the third layer and the second electrode, and a fourth layer between the second layer and the second electrode,
wherein the second layer comprises a light-emitting material,
wherein the fourth layer comprises a first organic compound,
wherein the first organic compound comprises a π-electron deficient heteroaromatic ring skeleton and a π-electron rich heteroaromatic ring skeleton,
wherein the first layer is in contact with the first electrode,
wherein the first layer comprises a second organic compound and a third organic compound,
wherein the third organic compound has an electron-accepting property with respect to the second organic compound, and
wherein the first layer has a resistivity higher than or equal to 1×10⁴ [Ω·cm] and lower than or equal to 1×10⁷ [Ω·cm].

2. The light-emitting device according to claim 1,
wherein the first organic compound has a first HOMO level, and
wherein the first HOMO level is higher than or equal to −6.0 eV and lower than or equal to −5.6 eV.

3. The light-emitting device according to claim 1, wherein the first organic compound comprises a diazine skeleton and the π-electron rich heteroaromatic ring skeleton.

4. The light-emitting device according to claim 1, wherein the first organic compound comprises the π-electron deficient heteroaromatic ring skeleton and a carbazole skeleton.

5. The light-emitting device according to claim 1,
wherein the first organic compound is represented by General Formula (G1): 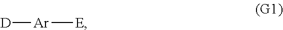
wherein D represents a substituted or unsubstituted quinoxalinyl group,
wherein E represents a substituted or unsubstituted carbazolyl group,
wherein Ar represents a substituted or unsubstituted arylene group, and
wherein the arylene group comprises 6 to 13 carbon atoms forming a ring.

6. The light-emitting device according to claim 1,
wherein the third organic compound has a LUMO level lower than or equal to −5.0 eV,
wherein the second organic compound has a second HOMO level, and
wherein the second HOMO level is higher than or equal to −5.7 eV and lower than or equal to −5.3 eV.

7. The light-emitting device according to claim 1,
wherein when a square root of electric-field strength is 600 [V/cm], the second organic compound has a hole mobility lower than or equal to 1×10⁻³ [cm²/Vs].

8. The light-emitting device according to claim 1,
wherein the first layer has a resistivity higher than or equal to 5×10⁴ [Ω·cm] and lower than or equal to 1×10⁷ [Ω·cm].

9. The light-emitting device according to claim 1,
wherein the first layer has a resistivity higher than or equal to 1×10⁵ [Ω·cm] and lower than or equal to 1×10⁷ [Ω·cm].

10. The light-emitting device according to claim 6,
wherein the third layer is held between the first layer and the second layer,
wherein the third layer is in contact with the first layer,
wherein the third layer comprises a fourth organic compound,
wherein the fourth organic compound has a third HOMO level,
wherein the third HOMO level differs from the second HOMO level by higher than or equal to −0.2 eV and lower than or equal to 0 eV.

11. A display apparatus comprising:
the light-emitting device according to claim 1; and
a second light-emitting device,
wherein the second light-emitting device is adjacent to the light-emitting device,
wherein the second light-emitting device comprises a third electrode and a fifth layer,
wherein a first gap is between the third electrode and the first electrode,
wherein the fifth layer is held between the third electrode and the second electrode,
wherein the fifth layer is in contact with the third electrode,
wherein the fifth layer comprises the second organic compound,
wherein a second gap is between the fifth layer and the first layer, and
wherein the second gap overlaps with the first gap.

12. A light-emitting apparatus comprising:
the light-emitting device according to claim 1; and
a transistor or a substrate.

13. A display apparatus comprising:
the light-emitting device according to claim 1; and
a transistor or a substrate.

14. A lighting device comprising:
the light-emitting apparatus according to claim 12; and
a housing.

15. An electronic device comprising:
the display apparatus according to claim 13; and
a sensor, an operation button, a speaker, or a microphone.
